(19) United States
(12) Patent Application Publication
(10) Pub. No.: US 2023/0329777 A1
(43) Pub. Date: Oct. 19, 2023

(54) TECHNIQUES FOR CONTROLLING AN IRREVERSIBLE ELECTROPORATION SYSTEM

(71) Applicant: AngioDynamics, Inc., Latham, NY (US)

(72) Inventors: Gordon SINGLE, Danville, CA (US); Robert E. Neal, II, Richmond, VA (US); David Warden, Belmont, CA (US)

(73) Assignee: AngioDynamics, Inc., Latham, NY (US)

(21) Appl. No.: 18/343,545

(22) Filed: Jun. 28, 2023

Related U.S. Application Data
(63) Continuation of application No. 16/162,953, filed on Oct. 17, 2018.
Application No. 16/162,953 is a continuation-in-part of application No. 15/565,625, filed on Oct. 10, 2017, now abandoned.
(60) Provisional application No. 62/573,424, filed on Oct. 17, 2017.
(60) Provisional application No. 62/145,581, filed on Apr. 10, 2015.
(60) Provisional application No. 62/151,513, filed on Apr. 23, 2015.
(60) Provisional application No. 62/173,538, filed on Jun. 10, 2015.

(51) Int. Cl. A61B 18/14; A61B 34/00; A61B 34/10

(52) U.S. Cl. CPC A61B 18/1477; A61B 18/1492; A61B 34/25; A61B 34/10; A61B 2018/00613

(57) ABSTRACT

An improved user interface system for an irreversible electroporation (ORE) system is provided. User interfaces are provided that dynamically display information provided by an operator or provided by the IRE system during setup, planning, and implementation stages of an IRE procedure in a more intuitive and efficient manner. As a result of being provided the user interfaces described herein, operators can plan and implement more effective IRE procedures to the benefit of a patient.

### CROSS-REFERENCE TO RELATED APPLICATIONS

This application is a continuation of U.S. patent application Ser. No. 16/162,953, filed on Oct. 17, 2018, which claims the benefit of U.S. Provisional Application No. 62/573,424, filed on Oct. 17, 2017, and is a continuation-in-part of U.S. patent application Ser. No. 15/565,625, filed Oct. 10, 2017, now Abandoned, which is a U.S. National Phase of PCT/US16/26998, filed Apr. 11, 2016, which claims the benefit of U.S. Provisional Application No. 62/145,581, filed Apr. 10, 2015, and claims the benefit of U.S. Provisional Application No. 62/151,513, filed Apr. 23, 2015, and claims the benefit of U.S. Provisional Application No. 62/173,538, filed Jun. 10, 2015. The content of each of which are incorporated herein by reference in their entireties.

### TECHNICAL FIELD

Embodiments generally relate to management of an irreversible electroporation (IRE) system. More particularly, embodiments relate to systems providing a user interface for planning, implementing, and monitoring an IRE procedure.

Conventional user interfaces for establishing and implementing various medical procedures, including irreversible electroporation (IRE), fail to present information to an operator in an efficient and effective manner. Accordingly, what is needed is a user interface system for an IRE system that presents and receives information in a more effective and intuitive manner for the operator, thereby increasing the effectiveness and usefulness of the IRE system.

### BRIEF DESCRIPTION OF THE DRAWINGS

FIG. 1 illustrates an exemplary block diagram of a user interface system.

FIG. 2 illustrates a first exemplary user interface of the user interface system of FIG. 1.

FIG. 3 illustrates a second exemplary user interface of the user interface system of FIG. 1.

FIG. 4 illustrates a third exemplary user interface of the user interface system of FIG. 1.

FIG. 5 illustrates a fourth exemplary user interface of the user interface system of FIG. 1.

FIG. 6 illustrates a fifth exemplary user interface of the user interface system of FIG. 1.

FIG. 7A illustrates a first exemplary portion of the user interface of FIG. 5.

FIG. 7B illustrates a second exemplary portion of the user interface of FIG. 5.

FIG. 8A illustrates a third exemplary portion of the user interface of FIG. 5.

FIG. 8B illustrates a fourth exemplary portion of the user interface of FIG. 5.

FIG. 9A illustrates a fifth exemplary portion of the user interface of FIG. 5.

FIG. 9B illustrates a sixth exemplary portion of the user interface of FIG. 5.

FIG. 10 illustrates a seventh exemplary portion of the user interface of FIG. 5.

FIG. 11 illustrates a sixth exemplary user interface of the user interface system of FIG. 1.

FIG. 12 illustrates a seventh exemplary user interface of the user interface system of FIG. 1.

FIG. 13 illustrates an eighth exemplary user interface of the user interface system of FIG. 1.

FIG. 14 illustrates a ninth exemplary user interface of the user interface system of FIG. 1.

FIG. 15 illustrates a tenth exemplary user interface of the user interface system of FIG. 1.

FIG. 16 illustrates a first exemplary portion of the user interface of FIG. 15.

FIG. 17 illustrates the user interface of FIG. 15 during pulse delivery.

FIG. 18 illustrates second exemplary portions of the user interface of FIG. 15.

FIG. 19 illustrates a third exemplary portion of the user interface of FIG. 15.

FIG. 20 illustrates a fourth exemplary portion of the user interface of FIG. 15.

FIG. 21 illustrates an eleventh exemplary user interface of the user interface system of FIG. 1.

FIG. 22 illustrates a twelfth exemplary user interface of the user interface system of FIG. 1.

FIG. 23 illustrates an embodiment of a logic flow for providing the user interfaces or portions thereof depicted in FIGS. 2-22.

FIG. 24 illustrates an embodiment of a storage medium.

### DETAILED DESCRIPTION

This disclosure presents various systems, components, and methods related to an irreversible electroporation (IRE) system and a user interface provided by the IRE system. Each of the systems, components, and methods disclosed herein provides one or more advantages over conventional systems, components, and methods.

Various embodiments provide techniques for controlling an IRE system using an improved user interface system for the IRE system. User interfaces are provided that dynamically display information provided by an operator or provided by the IRE system during setup, planning, and implementation stages of an IRE procedure in a more intuitive, user-friendly, and efficient manner as compared to prior art systems. As a result, an operator can plan and implement more effective IRE procedures to the benefit of a patient. Additionally, the user interfaces can provide real-time monitoring of various IRE pulse parameters, the ability for the operator to have more control over setting IRE pulse parameters and IRE treatment settings, and the ability to customize IRE procedure parameters.

Various embodiments provide an improved IRE system and an improved user interface for interacting with the IRE system. In various embodiments, user interfaces are provided that can dynamically display a pulse parameters table for textual display of numerical values indicating a voltage, a pulse length, a number of pulses, and a distance for an active probe pair of a user selected probe array. The pulse parameter table can further indicate a polarity of the active probe pair. The user interfaces can further provide a probe placement grid for graphical display of the user selected probe array in relation to a lesion zone and a target ablation zone. Each probe of the user selected probe array displayed in the probe placement grid can comprise a probe grid icon and a probe number, with the active probe pair of the user selected probe array indicated by a dashed line connecting the probe grid icons of the active probe pair. The polarity of the active probe pair can be indicated by an arrow of the dashed line pointing to a negative probe of the active probe pair. The distance between the active probe pair can be indicated by a distance value positioned over the dashed line and by a spacing between the probe grid icons of the active probe pair. Modification of values displayed in the pulse parameters table can be dynamically reflected in the graphical depiction of the user selected probe array in the probe grid array. In various embodiments, as an alternative to using one or more active probe pairs, the user interfaces can reflect use of a single insertion device (SID) that can include two electrodes spaced apart on a single probe.

In various embodiments, user interfaces are provided that can dynamically display a pulse generation table for textual display of numerical values indicating a voltage, a pulse duration, and a number of pulses for pulse delivery by an active probe pair of a user selected probe array. The user interfaces can further include a probe pair status grid for graphical display of the user selected probe array in relation to a lesion zone and a target ablation zone. Additionally, an electrical results chart can be provided by the user interfaces for graphical display of the pulse delivery by the active probe pair based on the numerical values for the voltage, the pulse duration, and the number of pulses for the active probe pair specified within the pulse generation table. The pulse generation table can be updated during and after implementation of the pulse delivery to numerically indicate data graphically depicted in the electrical results chart. Other embodiments are described and disclosed.

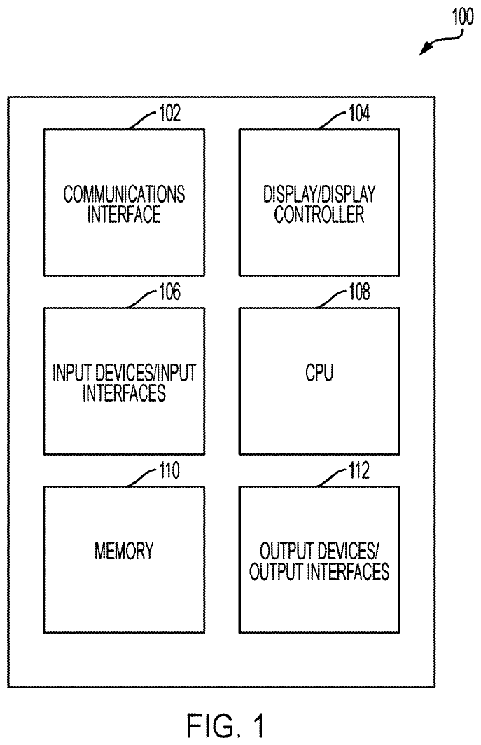

FIG. 1 illustrates a user interface system 100. The user interface system 100 can be coupled to and/or can be a component of a system that provides irreversible electroporation (IRE) and/or other electroporation-based therapies (EBTs) such as, for example, electrochemotherapy or electrogenetherapy. For purposes of discussion herein, the user interface system 100 can be considered to be coupled to and/or a component of an IRE system that can generate and deliver electric pulses through one or more probes to a patient for therapeutic purposes. Specifically, the IRE system can provide an ablation procedure that involves the delivery of a series of high voltage direct current electrical pulses between two electrodes placed within or around a targeted ablation area. The two electrodes can be positioned on a single probe—for example, on a SID—or can each be positioned on separate probes—for example, across two probes forming an active probe pair. In various embodiments, the IRE system can be the system disclosed in U.S. Pat. No. 9,078,665, which is incorporated herein by reference in its entirety.

The user interface system 100 can provide a user interface to a user or operator of the IRE system. As disclosed herein, the user interface provided by the user interface system 100 can enable the user to specify parameters for an IRE procedure, can enable the user to begin, pause, restart, or stop the IRE procedure, and can provide the user with real-time monitoring of the IRE procedure. As shown in FIG. 1, the user interface system 100 can include a communications interface 102, a display and a display controller 104, input devices and input device interfaces 106, a central processing unit (CPU), a processor, or a controller component 108, a memory component or unit 110, and output devices and output device interfaces 112.

The communications interface 102 can provide communication links between the user interface system 100 and one or more remote devices (not depicted in FIG. 1). The communications interface 102 can also provide communications links between the user interface system 100 and the IRE system. The communications interface 102 can provide communications over wired and/or wireless links according to any known wired or wireless communication standard or protocol. For example, the communications interface 102 can enable the user interface system 100 to communicate with one or more remote devices using, for example, Wi-Fi, a cellular communications standard, or Bluetooth. In various embodiments, the communications interface 102 can provide a communication link to any constituent component of the user interface system 100 that may be a remote component of the user interface system 100.

The display and display controller 104 can represent a visual display that can render visual information and a display controller for controlling the rendering of any visual information. The visual information can be any graphical or textual information. The display 104 can be a touchscreen or a touch-sensitive display. In various embodiments, the display 104 can be a wireless display and can be a remote component of the user interface system 100.

The input devices and input device interfaces 106 can represent any number of input devices and interfaces that can process any input provided through an input device. For example, the input devices 106 can include a mouse, a keyboard, a touchscreen, and a microphone. The input devices 106 can also include other devices of the IRE system such as, for example, a foot pedal and any other knobs, switches or user interaction components. The input device interfaces 106 can include one or more receivers for receiving input signals from any corresponding input device. In various embodiments, the user interface system 100 can receive information from a user by way of the input devices 106 including, for example, through voice commands.

The output devices and output devices interfaces 112 can represent any number of output devices and interfaces that can generate and provide outputs through an output device. For example, the output devices 112 can include visible, audible, or tactical (e.g., vibrational) outputs. In various embodiments, the output devices can include one or more probes used for a specified IRE procedure. The output device interfaces 112 can include one or more transmitters for generating and providing output signals from any corresponding output device.

The CPU or processor 108 can be a processor for executing instructions stored in the memory 110. The processor can control and direct operation of any of the components of the user interface system 100. In particular, the processor 108 can control the operation or functionality of the communications interface 102, the display/display controller 104, the input devices/input device interfaces 106, and the output devices/output device interfaces 112.

The communications interface 102, the display/display controller 104, the input devices/input device interfaces 106, and the output devices/output device interfaces 112 can be implemented in hardware, software, or any combination thereof. The user interface system 100 can include other modules, components, or devices implemented in hardware, software, or any combination thereof and not shown in FIG. 1 to facilitate communication with remote devices, the receiving of input signals from a user or the RE system, and the presentation of visual or audible information to the user.

As described herein, in various embodiments, the user interface system 100 can operate as part of an IRE system. For example, the user interface system 100 can provide a user interface that can be automatically and dynamically updated based on inputs provided by the user for setting parameters for an IRE procedure or based on information provided by the IRE system. The user interface provided by the user interface system 100 can also be automatically and dynamically updated based on real-time delivery of pulses generated and applied in accordance with input parameters provided by the user.

In various embodiments, the user interface system 100 can be coupled to and/or can be a portion of the IRE system described in U.S. patent application Ser. No. 15/565,625, filed Oct. 10, 2017, which is incorporated herein by reference in its entirety. The user interface system 100 can provide techniques for controlling the IRE system through the provided user interface, including establishing parameters for implementing an IRE procedure and controlling and/or monitoring the IRE procedure. In various embodiments, the user interface system 100 can provide a user interface that the user interacts with to establish parameters for a planned IRE procedure.

The parameters specified by the user can be stored (e.g., within the memory unit 110) and can be provided to the IRE system for implementation of the IRE procedure in accordance with the stored parameters. In various embodiments, pulses can be generated and delivered to the patient through one or more probes based on the stored parameters. In various embodiments, the user interface system 100 can provide a user interface that the user can use to monitor and/or control the IRE procedure being implemented based on the parameters set by the user. Accordingly, the IRE system can generate pulses for delivery to a patient through probes connected to the IRE system, with the pulses generated based on parameters set by the user through interaction with the user interface. The user interface further allows the user to initiate and monitor the pulse delivery. In particular, the user interface allows the user to define and modify pulse parameters and other IRE treatment parameters. Data regarding pulse delivery can be provided to the user interface system 100 and/or user interface during pulse delivery and upon completion. The user interface can be modified and/or updated based on this data to reflect real-time results of the pulse delivery for the user.

The user interface of the user interface system 100 can be provided by one or more of the components depicted in FIG. 1. The user interface can be provided based on input signals received from the communications interface 102 and the input devices/input interfaces 106. The display/display controller 104 can modify the user interface based on such received input signals. Further, the display/display controller 104 can retrieve data—such as graphics, icons, and text—from the memory 110 for display on the user interface based on the received input signals.

Each of the constituent components of the user interface system 100 can operate based on direction provided by the processor 108 to provide the user interface. For example, the user interface may include different operational modes. When a different mode is selected, the display/display controller 104 can retrieve different graphics from the memory 110 for presentation on the display 104. The different mode can be selected by the user through the touchscreen display 104 for example. Further, data received from a remote device (e.g., the IRE system) by way of the communications interface 102 can also be presented on the user interface. In various embodiments, the communications interface 102 can be coupled to various remote computing devices, databases (e.g., patient data databases), networks (e.g., a local hospital network), and/or cloud network or storage systems to allow remotely stored patient data to be provided to the user interface system 100 (e.g., automatically provided as input patient history). Exemplary features of the user interface provided by the user interface system 100 are described below.

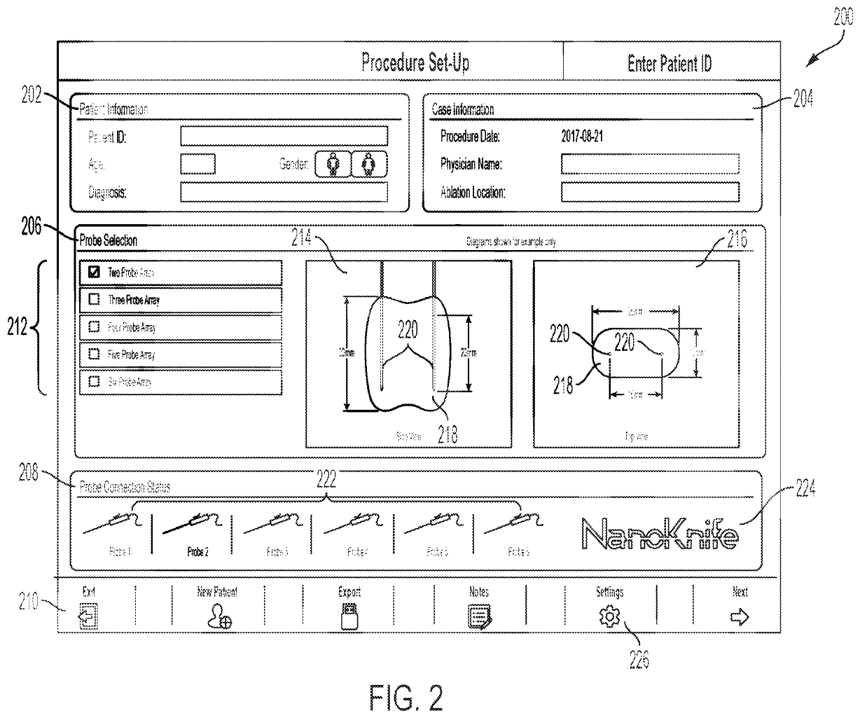

FIG. 2 illustrates a user interface 200. The user interface 200 can be provided by the user interface system 100. The user can provide and review information related to an IRE procedure through interaction with the user interface 200. In various embodiments, the user interface 200 can be considered to be a procedure setup user interface.

The user interface 200 can include various portions or panels including a patient information panel 202, a case information panel 204, a probe selection panel 206, and a probe connection status panel 208. The user interface 200 can further include an input panel 210 for the user to further direct operation of the user interface system 100.

The patient information panel 202 allows the user to enter or specify patient information. Data related to the patient can be entered, for example, through user interaction with a keyboard, a touchscreen, and/or a mouse. Gender can be selected and indicated by highlighting the gender related icons shown.

In various embodiments, for each of the user interfaces provided by the user interface system 100 and described herein, the user can interact with the provided user interface using any combination of keyboard entries, touchpad clicks, voice commands, touchscreen touches, and/or mouse movements and clicks. For simplicity, any reference to the user interacting with a provided user interface of the user interface system 100 can at least include any of these forms of user inputs. In various embodiments, any of the user interfaces provided by the user interface system 100 and described herein can be provided on a remote display 104 such as, for example, a tablet that can communicate wirelessly with the user interface system 100 through the communications interface 102.

The case information panel 204 allows the user to enter procedure information. The procedure date can be automatically set and shown. Other data related to the procedure can be entered, for example, through user interaction with any input device.

The probe selection panel 206 allows a user to select a probe type and a number of probes, referred to as a selected probe array. The probe selection panel 206 can include a list of probe types 212, a first image pane 214, and a second image pane 216. The first image pane 214 can display a side view of an ablation zone 218 (e.g., a side view of a size and shape of the ablation zone 218) relative to the number and position of probes 220 selected from the probe type list 212. The second image pane 216 can display a top view of the ablation zone 218 relative to the number and position of the probes 220 selected from the probe type list 212. The size and shape of the ablation zone 218 and the number and arrangement of the probes 220 in the first and second image panes 214 and 216 can be dynamically modified based on the user's selection from the probe type list 212. For example, the first and second image panes 214 and 216 can be updated based on selection of a different probe array from the probe type list 212.

The probe connection status panel 208 can include multiple probe icons 222 and a logo icon 224 (e.g., a name of the IRE system). Display of the probe icons 222 can vary based on a determined status of the number of probes connected to the IRE system. In various embodiments, one or more of the probe icons 222 can be displayed in a first manner to indicate a probe is not connected or not recognized; displayed in a second manner to indicate a first type of probe (e.g., an activation probe) is connected and valid, displayed in a third manner to indicate a second type of probe (e.g., a standard probe) is connected and valid; and displayed in a fourth manner to indicate a probe is connected and expired or invalid. In various embodiments, each manner of display can correspond to a different color to distinguish the different possible operational states for the probe icons 222. In various embodiments, any displayed portion of any user interface (e.g., any graphical or textual feature) provided by the user interface system 100 can vary in a manner of display by highlighting, changing a color (e.g., muting a color), changing a background color, and/or changing a size of any displayed icon, graphic, or text to indicate, for example, a change in value, a change in a condition, a selection made by the user, and/or a change in an operational state.

Display of the logo icon 224 can vary based on a determined status of whether probe connection requirements have been met. In various embodiments, the logo icon 224 can be displayed in a first manner to indicate that no probes are connected or the number of probes connected is less than the number of probes selected by the user in the probe type list 212; displayed in a second manner to indicate the number of probes connected matches the number of probes selected by the user in the probe type list 212 and that all other probe connection requirements have been met; and displayed in a third manner to indicate the number of probes connected exceeds the number of probes selected by the user in the probe type list 212 and/or that all other probe connection requirements have not been met. In various embodiments, each manner of display can correspond to a different color to distinguish the different possible operational states that can be indicated by the logo icon 224. Other variations in the manner of display as disclosed herein can also be provided.

The input panel 210, as shown in FIG. 2, allows the user to exit the first user interface 200, to provide information for another patient on another instance of the first user interface 200, to export data related to the user interface system 100, to enter reference notes for a planned procedure, to adjust settings related to the user interface system 100, and to advance to a next provided user interface of the user interface system 100. These operations can be made available to the user after clicking on the corresponding icon within the input panel such as, for example, a settings icon 226.

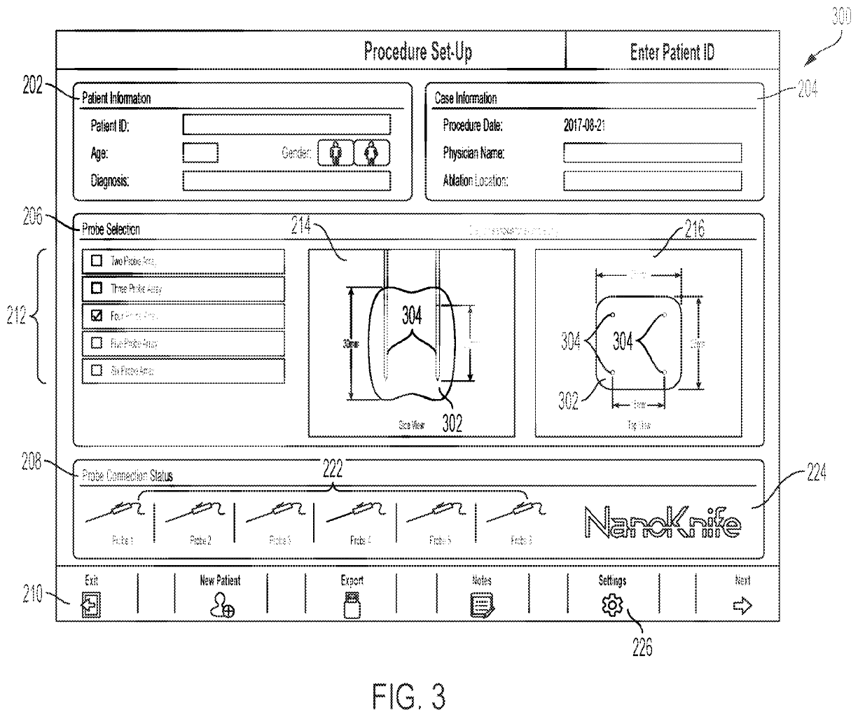

FIG. 3 illustrates a user interface 300. The user interface 300 can be provided by the user interface system 100. The user interface 300 can represent a variation of the user interface 200 when the user selects a probe array that is different from the probe array selected by the user in relation to the user interface 200. In various embodiments, the user interface 300 can also be considered to be a procedure setup user interface.

As shown in FIG. 3, the probe type list 212 can indicate that a four probe array has been selected (in contrast to the user interface 200 which shows that a two probe array has been selected). Accordingly, the first image pane 214 shows a side view of an ablation zone 302 and probes 304 corresponding to the selected probe array from the probe type list 212. The second image pane 216 shows a top view of the ablation zone 302 and the probes 304 in accordance with the selected probe array. A size and shape of the ablation zone 302 can vary from the size and shape of the ablation zone 218. The number and arrangement of the probes 304 can vary from the number and arrangement of the probes 220. FIGS. 2 and 3 illustrate the user interface system 100 dynamically updating the first and second image panes 214 and 216 based on the user's selection of the probe array from the probe type list 212.

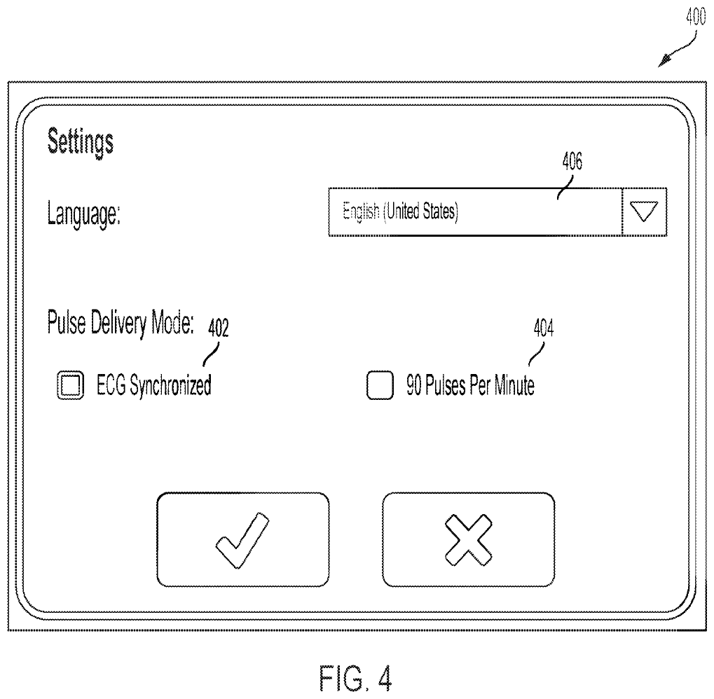

FIG. 4 illustrates a user interface 400. The user interface 400 can be provided by the user interface system 100. The user can change a pulse delivery mode through interaction with the user interface 400. In various embodiments, the user interface 400 can be considered to be a settings dialog box user interface presented to the user when the user selects the settings icon 226 on the input panel 210 displayed, for example, by the user interface 200 (or the user interface 300). The user interface 400 can display a selection for an ECG synchronized pulse delivery mode 402 and a selection for a 90 pulses per minute pulse delivery mode 404. The user can select between the ECG synchronized and 90 pulses per minute settings 402 and 404. A language setting 406 can also be selected by the user through interaction with the user interface 400. In various embodiments, the ECG synchronized pulse delivery mode 402 can be provided in accordance with synchronization techniques disclosed in U.S. Pat. No. 8,903,488, which is herein incorporated by reference in its entirety.

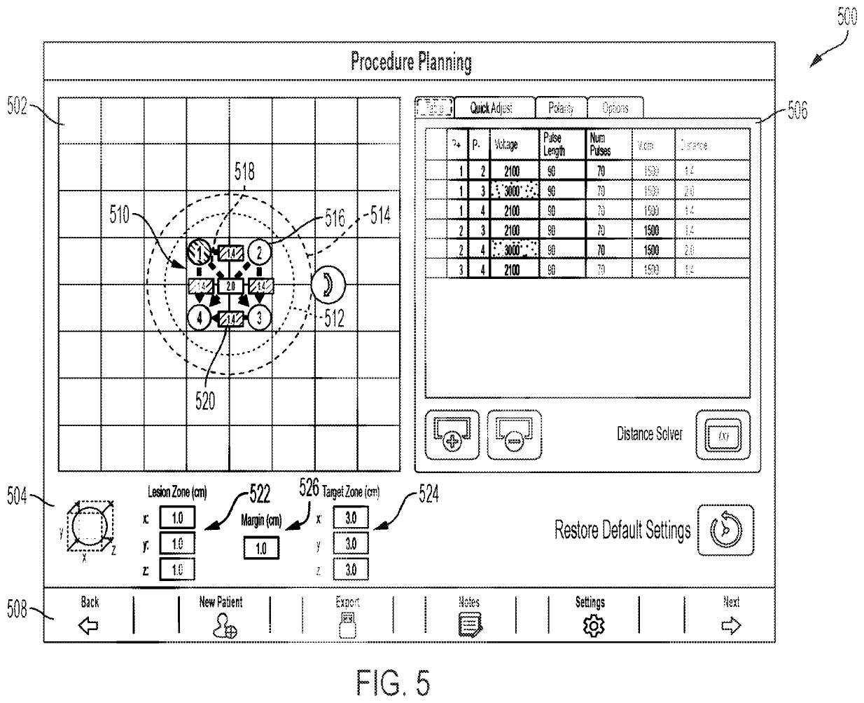

FIG. 5 illustrates a user interface 500. The user interface 500 can be provided by the user interface system 100. The user can plan probe placement and can define pulse parameters through interaction with the user interface 500. In various embodiments, the user interface 500 can be considered to be a procedure planning user interface.

The user interface 500 can include various portions or panels including a probe placement grid 502, a target ablation area settings area 504, and a parameters and options panel 506. The user interface 500 can also include an input panel 508 for the user to further direct operation of the user interface system 100. The user interface 500 can be presented to the user subsequent to the user interface 200 (or the user interface 300) after the user selects the next icon on the input panel 210. In various embodiments, the user interface 500 can provide the probe placement grid 502 in accordance with techniques disclosed in U.S. Pat. Nos. 9,198,733, 9,283,051, and U.S. patent application Ser. No. 15/239,229, filed Aug. 17, 2016, each of which is herein incorporated by reference in its entirety.

The probe placement grid 502 shows a selected probe array 510, a lesion zone 512, and a target ablation zone 514. The selected probe array 510 includes one or more probe grid icons 516. The number of probe grid icons 516 can correspond to the number of probes selected from the probe type list 212 on the user interface 200 (or the user interface 300). Each probe grid icon 516 can include a number to represent a probe with the selected probe array 510. As shown in FIG. 5, the selected probe array 510 can include four probes. Accordingly, the selected probe array 510 can include four probe grid icons 516 labeled “1” “2”, “3”, and “4”. In various embodiments, the number of probe grid icons 516 shown in the probe placement grid 502 can be automatically generated based on the number of probes selected by the user from the probe type list 212. As an example, the selected probe array 510 of the user interface 500 of FIG. 5 displays four probe grid icons 516 to correspond to the four probes 304 selected by the user in the probe type list 212 as shown in the user interface 300 of FIG. 3.

In various embodiments, the probe placement grid 502 can include vertical and horizontal grid lines as shown in FIG. 5. In various embodiments, the probe placement grid 502 can be an 8 cm×8 cm grid, with each grid box representing a 1 cm×1 cm grid cell (e.g., such that grid lines are spaced 1 cm apart). The vertical and horizontal grid lines shown in the probe placement grid 502 can be considered to be major grid lines. In various embodiments, the user interface 500 can allow the user to select to show minor grid lines (e.g., additional vertical and horizontal grid lines) between the major grid lines that are spaced 1 mm apart. The representation of the features shown in the probe placement grid 502—for example, the probe grid icons 516, the lesion zone 512, and the target ablation zone 514—are not necessarily reproduced or displayed according to scale.

The probe grid icons 516 displayed in the probe placement grid 502 can be virtual representations of the actual probes that will be used to deliver IRE treatment pulses. Display of each probe grid icon 516 can vary based on a connection status of the probe represented by a particular probe grid icon 516. In various embodiments, any of the probe grid icons 516 can be displayed in a first manner to indicate that the corresponding probe is not connected or recognized; can be displayed in a second manner to indicate that a corresponding probe is an activation probe and is connected and valid; can be displayed in a third manner to indicate the probe is a standard probe and is connected and valid; and displayed in a fourth manner to indicate that the corresponding probe is connected but is expired or invalid. In various embodiments, each manner of display can correspond to a different color to distinguish the different possible operation states represented by the probe grid icons 516.

Dashed lines 518 that connect two probe gird icons 516 can represent and indicate active probe pairs. The dashed lines 518 can include an arrow or arrowhead to indicate a polarity of an active probe pair. In various embodiments, the arrow of a dashed line 518 can point to a negative probe of an active probe pair. As an example, the probe grid icon 516 representing the first probe (labeled as “1”) can be a positive probe and the probe grid icon 516 representing the fourth probe (labeled as “4”) can be a negative probe in relation to the first probe. In this way, polarity between all active probes within the selected probe array 510 can be quickly and effectively conveyed to the user.

Distance indicators 520 can be positioned over the dashed lines 518 that indicate active probes. The distance indicators 510 can specify a distance between a probe pair (e.g., in centimeters). As an example, the distance indicator 510 between the probe grid icon 516 representing the first probe (labeled as “1”) and the probe grid icon 516 representing the fourth probe (labeled as “4”) can indicate a distance of 1.4 centimeters. As a further example, the distance indicator 510 between the probe grid icon 516 representing the first probe (labeled as “1”) and the probe grid icon 516 representing the third probe (labeled as “3”) can indicate a distance of 2.0 centimeters. In this way, distances between probe pairs within the selected probe array 510 can be quickly and effectively conveyed to the user.

The target ablation area settings panel 504 can include a lesion zone text box area 522, a margin text box area 526, and a target zone text box area 524. The lesion zone 512 can be represented as a circular area within the probe placement grid 502. The target ablation zone 514 can surround the lesion zone 512 by a set distance referred to as a margin. For example, a distance between an outer perimeter of the lesion zone 512 and an outer perimeter of the target ablation zone 514 can be the margin. In various embodiments, a size of the target ablation zone 514 can be determined based on the user providing a size of the lesion zone 512 through the lesion zone text box area 522 and providing a size of the margin through the margin text box area 526. The user interface 500 can subsequently update the displayed sizes of the lesion zone 512 and the target ablation zone 514 within the probe placement grid 502.

The lesion zone text box area 522 can include three text boxes that can represent the three corresponding diameters of the lesion zone 512 along three-dimensional orthogonal axes—for example, X, Y, and Z coordinate axes, with each axis being perpendicular to the other two axes. The X and Y text box values can represent the width and height diameters, respectively, of the lesion zone 512 that are perpendicular to an anticipated probe placement trajectory. The Z text box value can represent the diameter of the lesion zone 512 that runs along the anticipated probe placement trajectory (e.g., parallel to the anticipated probe placement trajectory). The margin text box area 526 can include a single text box that can represent the margin between the lesion zone 512 and the target ablation zone 514.

The target ablation area settings panel 504 allows the user to specify the diameters of the lesion zone 512 and the margin between the lesion zone 512 and the target ablation zone 514 using the lesion zone text box area 522 and the margin text box area 526, respectively. Based on these provided values, the user interface system 100 can automatically determine the target ablation zone 514. The graphical representations of the lesion zone 512 and the target ablation zone 514 (and therefore the margin therebetween) can be dynamically updated within the probe placement grid 502 based on inputs provided by the user.

The parameters and options panel 506 can allow the user to modify pulse parameters and to adjust features of the probe placement grid 502 as described further herein, in various embodiments, each individual probe grid icon 516 can be moved in relation to the other displayed probe grid icons 516. In doing so, the user interface 500 can dynamically update the distance indicators 520. Further, in various embodiments, the user interface 500 can allow the user to move the entire selected probe array 510 in its entirety as the target ablation zone 514 and the lesion zone 512 are displayed in fixed positions.

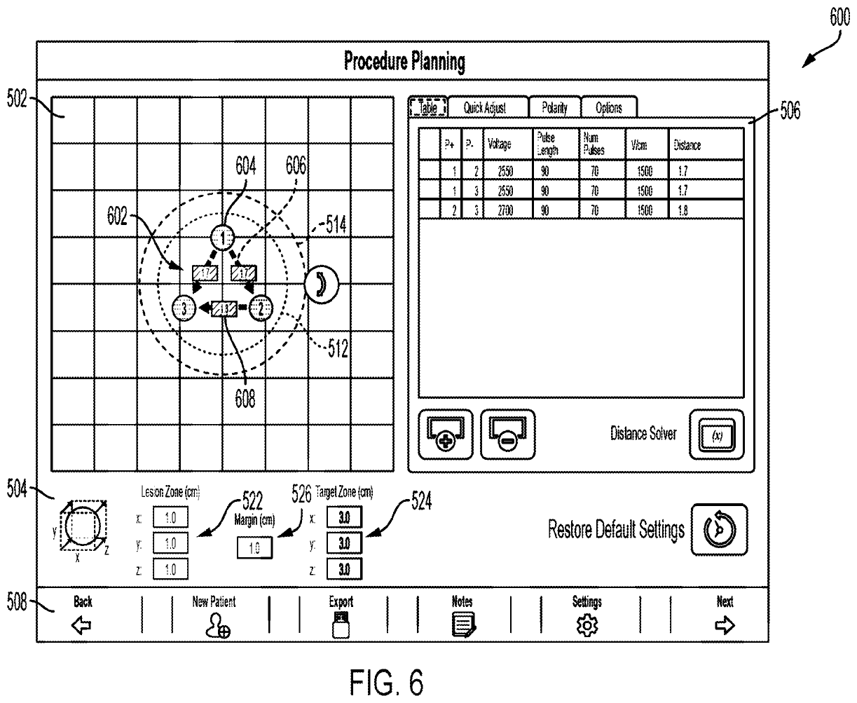

FIG. 6 illustrates a user interface 600. The user interface 600 can be provided by the user interface system 100. The user interface 600 can represent a variation of the user interface 500 and can indicate a probe selection that is different from the probe selection displayed by the user interface 500. In various embodiments, the user interface 600 can also be considered to be a procedure planning user interface.

As shown in FIG. 6, the probe placement grid 502 includes a selected probe array 602. The selected probe array 602 includes three probe grid icons 604, in contrast to the user interface 500 which shows four probe grid icons 516 in the selected probe array 510. The number of probe grid icons 604 can correspond to the number of probes selected from the probe type list 212 on the user interface 200 (or the user interface 300). As an example, the selected probe array 602 includes three probe grid icons 604 labeled “1”, “2”, and “3”.

As similarly shown in the user interface 500, dashed lines 606 that connect two probe grid icons 604 can represent active probe pairs and the arrow portion of a dashed line 606 can indicate a negative probe of an active probe pair. As an example, the probe grid icon 604 representing the first probe (labeled as “1”) can be a positive probe and the probe grid icon 516 representing the third probe (labeled as “3”) can be a negative probe in relation to the first probe. In this way, polarity between all active probes within the selected probe array 602 can be quickly and effectively conveyed to the user.

Further, as also similarly shown in the user interface 500, distance indicators 608 can be positioned over the dashed lines 606 and can specify a distance between a probe pair (e.g., in centimeters). As an example, the distance indicator 608 between the probe grid icon 604 representing the first probe (labeled as “1”) and the probe grid icon 604 representing the third probe (labeled as “3”) can indicate a distance of 1.7 centimeters. In this way, distances between probe pairs within the selected probe array 602 can be quickly and effectively conveyed to the user.

The user interfaces 500 and 600 demonstrate the user interface system 100 efficiently representing the number of probes selected by the user, the active probe pairs and their polarities, and the distances between the probes in relation to the area being treated.

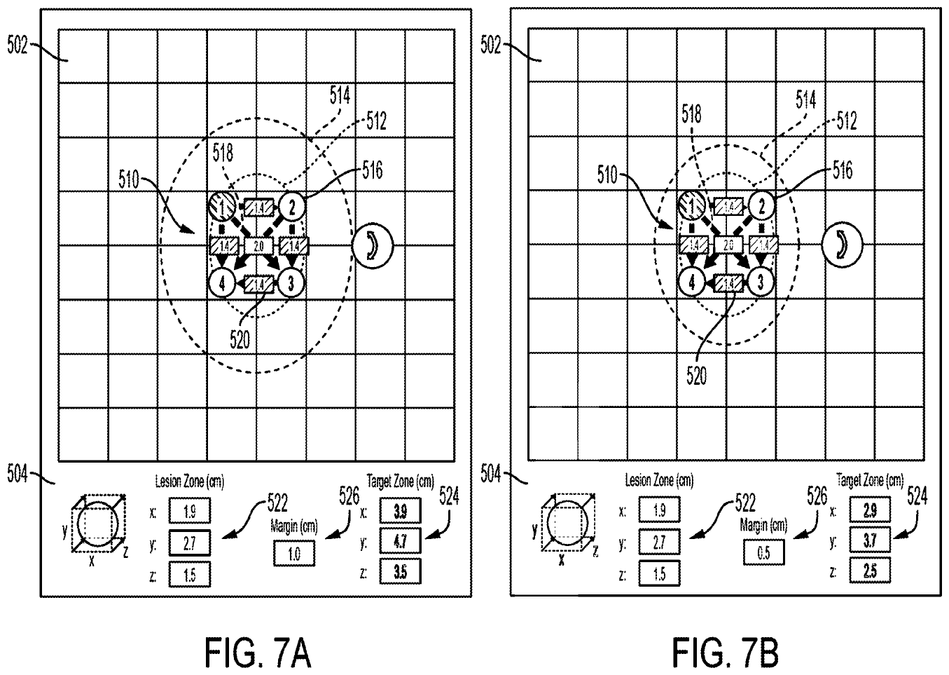

FIGS. 7A and 7B illustrate dynamic updating of the size of the lesion zone 512 and the target ablation zone 514 as shown in the probe placement grid 502 based on input from the user. Specifically, FIG. 7A shows a first relationship between the lesion zone 512 and the target ablation zone 514 when a first margin value (e.g., 1 cm) is specified in the margin text box area 526. As shown in the probe placement grid 502, the distance between the lesion zone 512 and the target ablation zone 514 corresponds to the value specified in the margin text box area 526.

FIG. 7B shows a second relationship between the lesion zone 512 and the target ablation zone 514 when a second margin value (e.g., 0.5 cm) is specified in the margin text box area 526. As shown, the distance between the lesion zone 512 and the target ablation zone 514 corresponds to the value specified in the margin text box area 526. In comparison to FIG. 7A, the distance between the lesion zone 512 and the target ablation zone 514 is smaller in FIG. 7B to correspond to the smaller margin value in the margin text box area 526 specified within the target ablation area settings panel 504. Additionally, values corresponding to the size of the target ablation zone 514 as displayed in the target zone text box area 524 can be automatically updated based on the user changing the value specified in the margin text box area 526. FIGS. 7A and 7B demonstrate the user interface system 100 dynamically displaying differently sized target ablation zones 514 within the probe placement grid 502 of the user interface 500 (or the user interface 600) based on user input as well as dynamically updating values displayed in the target zone text box area 524 based on user changes to the value displayed in the margin text box area 526.

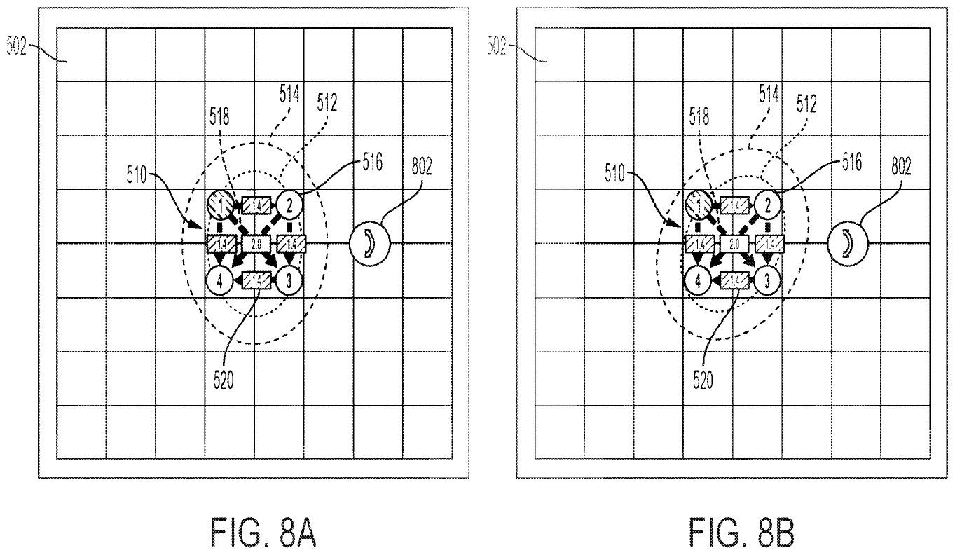

FIGS. 8A and 8B illustrate dynamic rotation of the lesion zone 512 and the target ablation zone 514 as shown in the probe placement grid 502 based on input from the user. Specifically, FIG. 8A shows a first orientation of the lesion zone 512 and the target ablation zone 514 within the probe placement grid 502. A first implementation of a target zone rotation handle icon 802 can indicate that the lesion zone 512 and the target ablation zone 514 are in a rotation mode. Accordingly, the user can rotate the lesion zone 512 and the target ablation zone 514 by providing a corresponding input (e.g., by using a mouse or a touchscreen or any of the other user input mechanisms described herein). The lesion zone 512 and the target ablation zone 514 can be rotated up to 360 degrees in a clockwise or a counter-clockwise direction. Based on the click and dragging input from the user, the target ablation zone 514 can rotate about a center point of the lesion zone 512 in the same direction as the click and drag motion inputted by the user.

FIG. 8B shows a second orientation of the lesion zone 512 and the target ablation zone 514 within the probe placement grid 502 (e.g., after input from the user). The lesion zone 512 and the target ablation zone 514 are shown rotated together in comparison to the orientation of the lesion zone 512 and the target ablation zone 514 within the probe placement grid 502 shown in FIG. 8A. FIGS. 8A and 8B demonstrate the user interface system 100 dynamically displaying different rotational orientations of the lesion zone 512 and the target ablation zone 514 within the probe placement grid 502 of the user interface 500 (or the user interface 600) based on user input.

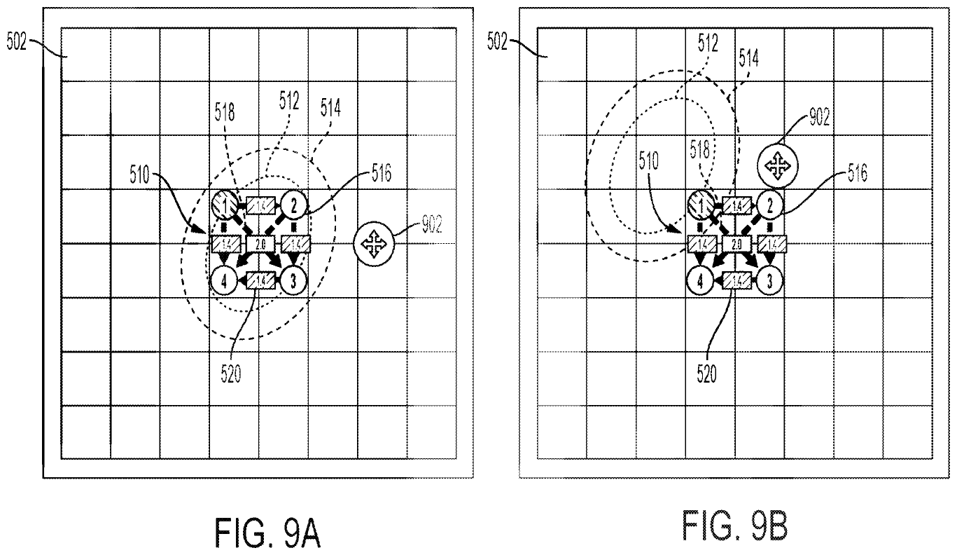

FIGS. 9A and 9B illustrate dynamic translation of the lesion zone 512 and the target ablation zone 514 as shown in the probe placement grid 502 based on input from the user. Specifically, FIG. 9A shows a first positioning of the lesion zone 512 and the target ablation zone 514 within the probe placement grid 502. A second implementation of the target zone rotation handle icon 802 can indicate that the lesion zone 512 and the target ablation zone 514 are in a translation mode. In various embodiments, the user can provide an input (e.g., a right mouse click) to toggle between rotation of the lesion zone 512 and the target ablation zone 514 and translation of the lesion zone 512 and the target ablation zone 514. The target zone rotation handle icon 802 can be displayed as shown in FIGS. 8A and 8B to indicate a rotation mode and, based on a particular user input, can be displayed as a target zone translation handle icon 902 as shown in FIGS. 9A and 9B to indicate a translation mode.

After engaging the translation mode, the user can move the lesion zone 512 and the target ablation zone 514 (e.g., by using a mouse, a touchscreen, or any user interaction mechanisms described herein). The lesion zone 512 and the target ablation zone 514 can be moved together based on user input within the probe placement grid 502. If the user is working with a particularly complex probe array and the inter-probe distances between the probe grid icons 516 is confirmed with an imaging method, but the probe grid icons 516 are off center from the lesion zone 512 and target ablation zone 514, the user can drag both of the lesion zone 512 and target ablation zone 512 in order to be centered within the probe grid icons 516. FIGS. 9A and 9B demonstrate the user interface system 100 dynamically displaying different translation orientations of the lesion zone 512 and the target ablation zone 514 within the probe placement grid 502 of the user interface 500 (or the user interface 600) based on the user input.

In various embodiments, the user interface system 100 can allow the user to adjust the position of any individual probe grid icon 516 or any group of probe grid icons 516. In doing so, distance indicators 520 can be dynamically updated along with textual display of distance information provided in parameters and options panel 506.

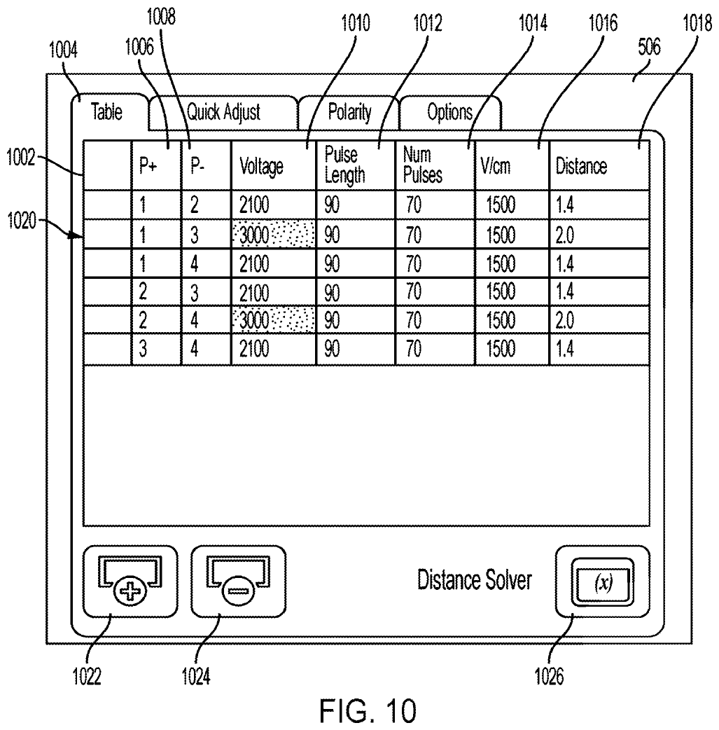

FIG. 10 illustrates a pulse parameters table 1002 displayed within the parameters and options panel 506 of the user interface 500 (or the user interface 600). The pulse parameters table 1002 can be located on and can be accessible by selecting a table tab 1004. The pulse parameters table 1002 can display default pulse parameters for the probe array selected by the user from the probe type list 212 on the user interface 200 (or the user interface 300). The pulse parameters table 1002 can indicate which probes are active probes, which can be probe pairs that deliver electrical pulses between a first probe in the pair to a second probe in the pair. In various embodiments, each row of the pulse parameters table 1002 can represent an active probe pair. In various embodiments, the default pulse parameters displayed within the pulse parameters table 1002 can be stored in the memory 110. In various embodiments, the displayed default pulse parameters can relate to and can vary based on a tissue type and/or a treatment type specified by the user. As an example, a first set of default pulse parameters can be displayed within the pulse parameters table 1002 when the user selects a first type of tissue for treatment (e.g., liver tissue) and a second set of default pulse parameters can be displayed within the pulse parameters table 1002 when the user selects a second type of tissue for treatment (e.g., pancreas tissue).

The pulse parameters table 1002 can include a number of columns. A first column 1006 can indicate a positive probe of an active probe pair. A second column 1008 can indicate a negative probe of an active probe pair. A third column 1010 can indicate a maximum voltage (e.g., in volts) of each pulse to be delivered between an active probe pair. A fourth column 1012 can indicate a duration of each pulse to be delivered between an active probe pair (e.g., in microseconds). A fifth column 1014 can indicate an intended number of pulses to be delivered between an active probe pair. A sixth column 1016 can indicate a voltage gradient of a planned treatment. In various embodiments, the voltage gradient can be a factor expressed in volts per centimeter that when multiplied by a probe pair distance provides an expected voltage of the active probe pair. A seventh column 1018 can indicate a distance (e.g., in centimeters) between a negative probe and a positive probe of an active probe pair.

As an example, a row 1020 can represent an active probe pair formed between a first probe (represented by “1” in the first column 1006) and a third probe (represented by “3” in the second column 1008). As shown in the pulse parameters table 1002, the first probe can be the positive probe and the third probe can be the negative probe. Further, the fifth column 1014 can indicate that 70 pulses are intended to be applied between the first and third probes, with the fourth column indicating that each pulse is to last 90 microseconds, and with the third column 1010 indicating that a maximum voltage of 3000 volts is intended for each pulse. Further, the seventh column 1018 of the pulse parameters table 1002 can indicate that the first and third probes are 2.0 centimeters apart.

Each parameter or value displayed within the pulse parameters table 1002 can be adjusted or modified by the user. As an example, a displayed parameter can be modified by the user directly entering a value for a particular parameter into a cell of the pulse parameters table 1002. As another example, a displayed parameter can be modified by using a pop-up window provided by the user interface system 100 for adjusting any parameter value. In various embodiments, the user interface system 100 can provide pop-up windows that allow any parameter modification for a particular column and row to be applied to all rows (e.g., each probe pair). In various embodiments, the distance between any active probe pair as indicated in column 1018 can be modified directly by the user or can be automatically updated based on orientation and/or location of the probe grid icons 516 within the probe placement grid 502.

In various embodiments, parameters specified in any cell of the pulse parameters table 1002 can be displayed in different manners to indicate a different status or condition of the parameter. For example, parameters can be displayed in a first manner (e.g., with a first background or cell backfill color) to indicate that a pulse parameter is currently set to a default value. Parameters can be displayed in a second manner (e.g., with a second background or cell backfill color) to indicate that a pulse parameter is currently set to a value that is above or below the default value. Parameters can be displayed in a third manner (e.g., with a third background or cell backfill color) to indicate that a pulse parameter is currently set to a maximum or a minimum value. In various embodiments, the pulse parameters table 1002 can initially include default values for certain pulse parameters displayed in the pulse parameters table 1002 that can be subsequently updated or modified by the user.

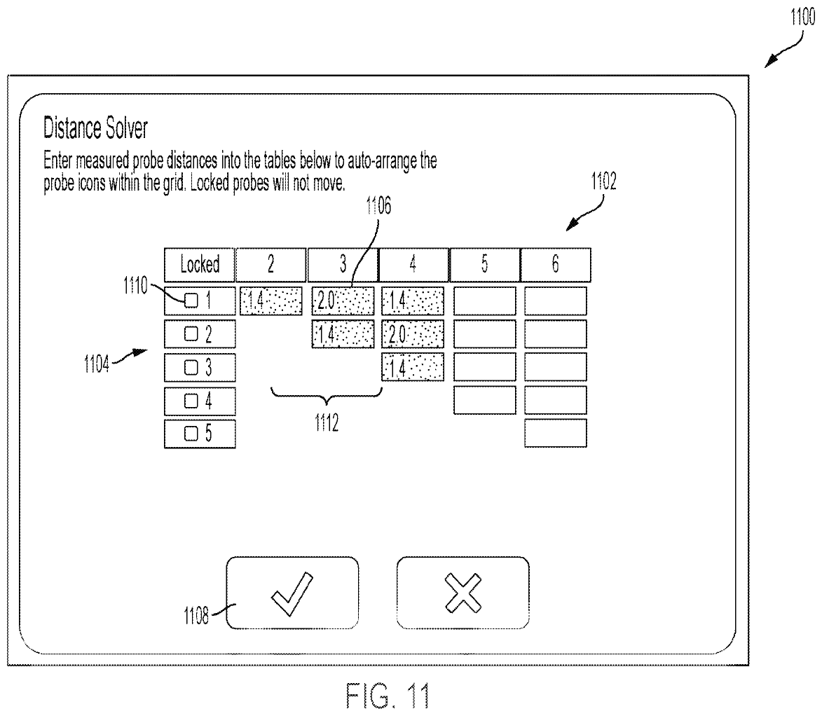

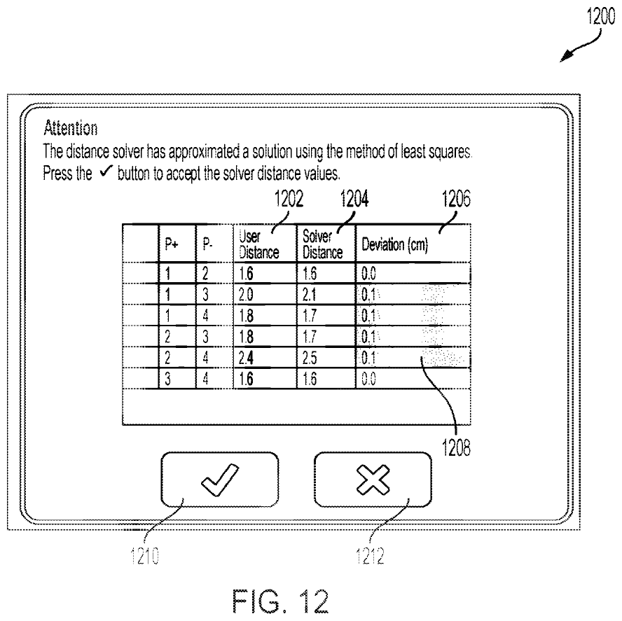

As further shown in FIG. 10, a first icon 1022 can be selected by the user to add a row to the pulse parameters table 1002—i.e., to add a new probe pair. A second icon 1024 can be selected by the user to delete a row of the pulse parameters table 1002—i.e., to delete a current probe pair. In this way, active probe pairs can be effectively managed. Adding or deleting probe pairs within the pulse parameters table 1002 can be dynamically reflected graphically within the probe placement grid 502. For example, distance indicators 520 and/or dashed lines 518 can be added to the probe placement grid 502 when a new probe pair is added to the pulse parameters table 1002 and certain distance indicators 520 and/or dashed lines 518 can be deleted from the probe placement grid 502 when a corresponding probe pair is deleted from the pulse parameters table 1002. A third icon 1026 can be selected by the user to activate a distance solver utility, described herein in relation to FIGS. 11 and 12.

As shown in relation to FIGS. 5 and 10, the pulse parameters table 1002 can textually display numerical values for various pulse parameters for each active probe pair. The pulse parameters table 1002 can also indicate a polarity of each active probe pair. The pulse parameters table 1002 can include such information for each probe pair within the selected probe array 510 specified by the user. Correspondingly, the probe placement grid 502 can graphically display the user selected probe array 510 along with certain pulse parameters textually displayed in the pulse parameters table 1002. The user interface system 100 can update the graphical display of the probe placement grid 502 (and its contents) based on user manipulation of any value textually displayed in the pulse parameter table 1002, including any change to the polarity of an active probe pair.

Similarly, numerical values displayed in the pulse parameter table 1002, can be updated based on user manipulation of the probe placement grid 502 and any of its contents. In this way, the pulse parameters table 1002 and the probe placement grid 502 can be updated based on changes to the other, to reflect pulse parameters for pulses to be generated and delivered by each active probe pair. This relationship between the pulse parameters table 1002 and the probe placement grid 502 can improve the experience of the operator of the user interface system 100 to provide improved IRE procedure planning.

FIG. 11 illustrates a user interface 1100. The user interface 1100 can be provided by the user interface system 100. The user interface 1100 can be displayed to the user when the user selects the third icon 1026 from the pulse parameters table 1002. The user interface 1100 can display a distance solver utility (and can be considered to be a distance solver utility user interface). The user interface 1100 allows the user to enter probe distances which are then used to automatically adjust the positioning of the individual probe icons 516 within the probe placement grid 502. The user interface 1100 allows the user to enter probe distances, for example, measured from an imaging device rather than using an input device (e.g., a touchpad or touchscreen) to manually move probes (e.g., the individual probe icons 516) on the probe placement grid 502.

As shown in FIG. 11, the user interface 1100 can display a row 1102 and a column 1104 defining possible active probe pairs. The user can enter distances for any active probe pair. As an example, the user can enter or modify a distance shown in a cell 1106 indicting a distance between probe 1 and probe 3. Cells 1112 can indicate all active probe pair combinations that can be modified by the user and can be displayed in a different manner (e.g., with a different background or cell backfill color) from cells of probe pair combinations that are inactive.

Distances for active probe pairs can be entered, for example, directly by the user or through a pop-up window. The user can select an icon 1108 to save the entered distances. The user interface 1100 allows the user to lock the position of a probe by selecting a lock box indicator 1110 that corresponds to a particular probe. When locked, a corresponding probe will not move (e.g., on the probe placement gird 502). Adjustments made by the user through interaction with the user interface 1100 can be dynamically updated for display on the probe placement grid 502 by the user interface system 100.

FIG. 12 illustrates a user interface 1200. The user interface 1100 can be provided by the user interface system 100. The user interface 1200 can be displayed to the user after the user selects the icon 1108 on the user interface 1100. The user interface 1200 can display a distance solver results dialog box (and can be considered to be a distance solver results user interface). For each active probe pair, the user interface 1200 can display distances inputted by the user 1202, distance solver calculated distances 1204, and any deviation 1206 between the distances inputted by the user 1202 and the distance solver calculated distances 1204. The distance solver calculated distances 1204 can represent distances between any probe pair calculated by the distance solver utility based on the distances inputted by the user 1202. In various embodiments, the distance solver calculated distances 1204 can provide a check of the distances inputted by the user 1202. Any conflict (e.g., differences) between values inputted by the user 1202 and distances automatically calculated by the distance solver utility 1204 can be displayed in the deviations column cells 1206.

In various embodiments, the distance solver calculated distances 1204 can be determined based on a least squares calculation (e.g., based on one or more of the distances inputted by the user 1202). Any non-zero deviation displayed in a deviations cell 1206 can be indicated by displaying a corresponding cell in a different manner (e.g., a different color or cell back fill color) from a deviations cell 1206 where no deviation is determined. As an example, cell 1208 can be displayed in a different color from other cells in the displayed array to indicate that the distance inputted by the user 1202 for probes 2 and 4 deviates from the distance solver calculated distances 1204 for probes 2 and 4. The user can either accept the distance solver utility results shown in column 1206—for example, by selecting an icon 1210—or can reject the distance solver utility results—for example, by selecting an icon 1212. The individual probe grid icons 516 can be arranged on the probe placement grid 502 based on the results selected by the user from the user interface 1200.

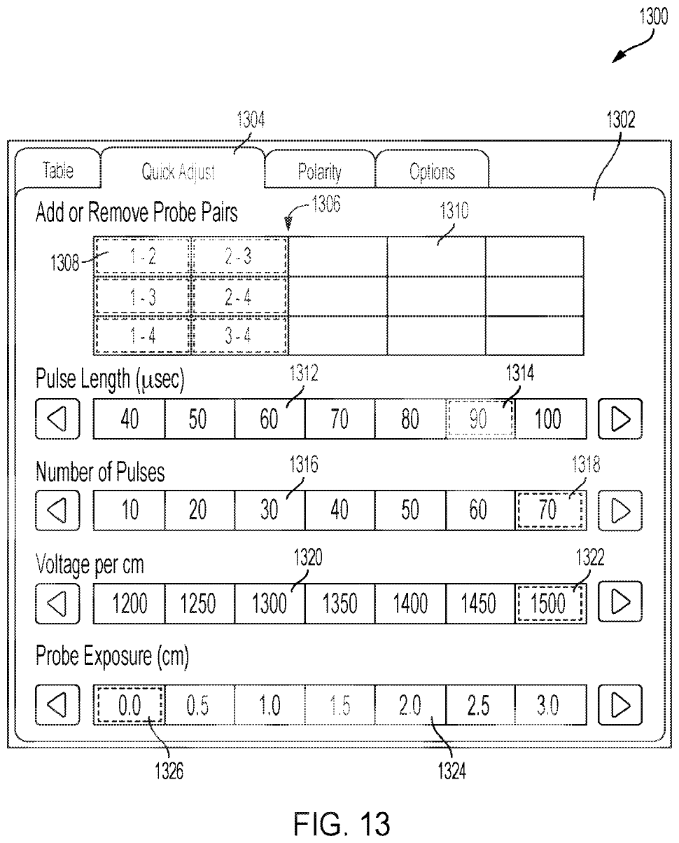

FIG. 13 illustrates a user interface 1300. The user interface 1300 can be provided by the user interface system 100. The user interface 1300 can display a quick adjust dialog box 1302 within the parameters and options panel 506 of the user interface 500 (or the user interface 600). In various embodiments, the user interface 1300 can be considered to be a quick adjust dialog box user interface. The quick adjust dialog box 1302 can be located on and can be accessible by selecting a quick adjust tab 1304. The quick adjust dialog box 1302 can allow the user to quickly and effectively add or remove probe pairs, modify pulse parameters for all active probe pairs, and to enter probe exposure settings. As a result, the user interface 1300 provides a user-friendly display of treatment parameters that allows for enhanced treatment customization.

As shown in FIG. 13, the user interface 1300 can display a table 1306 that can include cells of a first type 1308 that indicate pairs of active probes and cells of a second type 1310 that indicate inactive probe pairs. The first type of cells 1308 can be displayed in a different manner (e.g., a different color or cell back fill color) than the second type of cells 1310. The first type of cells 1308 can include a cell for each active probe pair as specified in the pulse parameters table 1002. When the user selects a cell from the first type of cells 1308, the corresponding active pair of probes can be removed (e.g., rendered inactive). When the user selects a cell from the second type of cells 1310, a corresponding possible active pair of probes can be added (e.g., rendered active).

The quick adjust dialog box 1302 allows a user to modify pulse length for all active probe pairs—specifically, each probe pair listed in the table 1306 (e.g., listed within a cell of the first type 1308). To do so, the user can select a pulse length value from a displayed value bar 1312. A current selected value 1314 can be indicated by displaying the current selected value 1314 in a different manner (e.g., a different color or cell back fill color) than the other available values displayed on the displayed value bar 1312. When the user selects a new value different from the current selected value 1314, the pulse length value for all active probe pairs can be set to the new value and automatically updated within the pulse parameters table 1002.

The quick adjust dialog box 1302 allows a user to modify the number of pulse for all active probe pairs—specifically, each probe pair listed in the table 1306 (e.g., listed within a cell of the first type 1308). To do so, the user can select a number of pulses value from a displayed value bar 1316. A current selected value 1318 can be indicated by displaying the current selected value 1318 in a different manner than the other available values displayed on the displayed value bar 1316. In various embodiments, the different manner of display can include, but is not limited to, changing a color of displayed text or changing a color of a displayed cell back fill color, or any of the other techniques for adjusting a manner of display disclosed herein. When the user selects a new value different from the current selected value 1318, the number of pulses value for all active probe pairs can be set to the new value and automatically updated within the pulse parameters table 1002.

The quick adjust dialog box 1302 allows a user to modify voltage per distance for all active probe pairs—specifically, each probe pair listed in the table 1306 (e.g., listed within a cell of the first type 1308). To do so, the user can select a voltage per distance factor (e.g., per cm) from a displayed value bar 1320. A current selected value 1322 can be indicated by displaying the current selected value 1322 in a different manner (e.g., a different color or cell back fill color) than the other available values displayed on the displayed value bar 1320. When the user selects a new value different from the current selected value 1322, the voltage per distance factor for all active probe pairs can be set to the new value and automatically updated within the pulse parameters table 1002.

The quick adjust dialog box 1302 allows a user to modify probe exposure for all active probe pairs—specifically, each probe pair listed in the table 1306 (e.g., listed within a cell of the first type 1308). To do so, the user can select a probe exposure value from a displayed value bar 1324. A current selected value 1326 can be indicated by displaying the current selected value 1326 in a different manner (e.g., a different color or cell back fill color) than the other available values displayed on the displayed value bar 1324. In various embodiments, probe exposure (e.g., a measure of a portion of a probe within a target zone) may not be displayed within the pulse parameters table 1002.

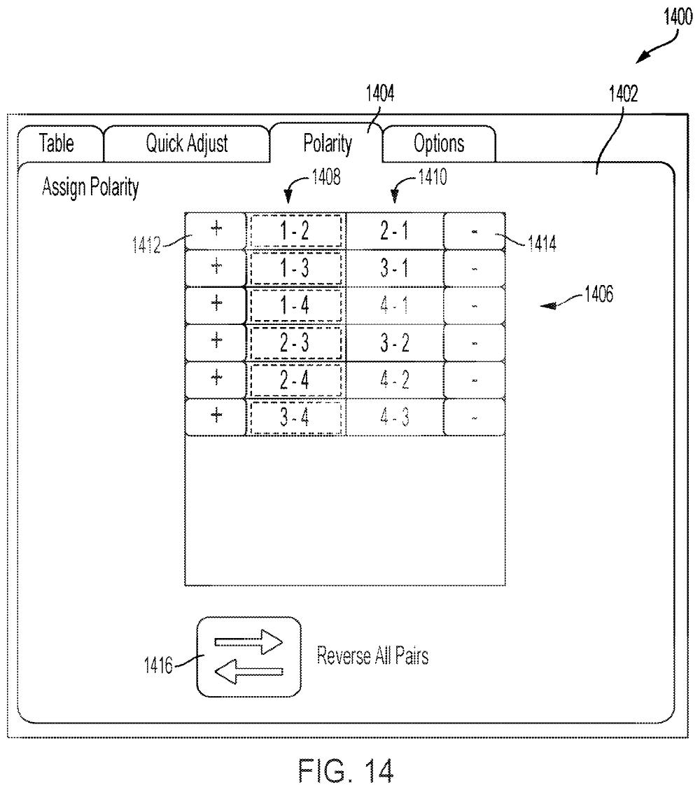

FIG. 14 illustrates a user interface 1400. The user interface 1400 can be provided by the user interface system 100. The user interface 1400 can display a polarity dialog box 1402. The polarity dialog box 1402 can be shown within the parameters and options panel 506 of the user interface 500 (or the user interface 600). In various embodiments, the user interface 1400 can be considered to be a polarity dialog box user interface. The polarity dialog box 1402 can be located on and can be accessible by selecting a polarity tab 1404. The polarity dialog box 1402 can allow the user to quickly re-assign the polarity of each probe pair individually or all probe pairs simultaneously.

As shown in FIG. 14, the user interface 1400 can display a table 1406. Each row of the table 1406 can represent an active probe pair (e.g., as shown in and corresponding to the pulse parameters table 1002). The table 1406 can display a current polarity assignment 1408 for each active probe pair and an alternative polarity assignment 1410 for each active probe pair. The currently polarity assignment 1408 for a given active probe pair can be displayed in a different manner (e.g., in a different color or cell backfill color) from a manner of displaying the alternative polarity assignment 1410 for the given active probe pair. The user can change a polarity assigned to a specific active probe pair by selecting a first icon 1412 or a second icon 1414, as appropriate, positioned next to the active probe pair. Alternatively, the user can change the polarity assigned to a specific active probe pair by selecting the alterative polarity assignment 1410. A third icon 1416 can be selected to switch the polarity assignment for all active probe pairs.

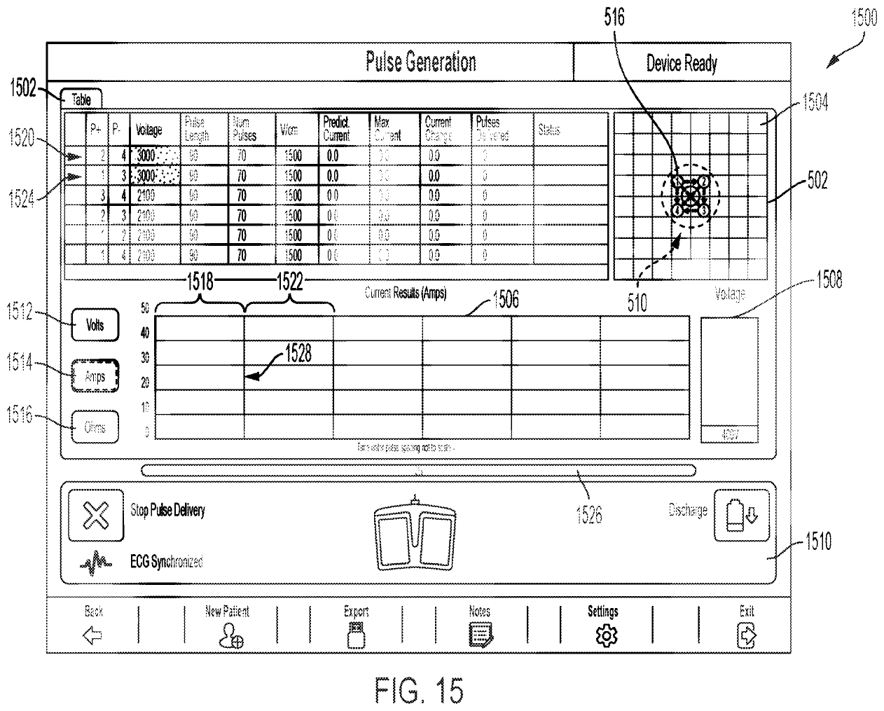

FIG. 15 illustrates a user interface 1500. The user interface 1500 can be provided by the user interface system 100. The user can control and monitor pulse delivery through interaction with the user interface 1500. In various embodiments, the user interface 500 can be considered to be a pulse generation user interface. The user interface 1500 can include various portions or panels including a pulse generation table 1502, a probe pair status grid 1504, an electrical results chart 1506, a voltage gauge 1508, and a pulse delivery control panel 1510. The user interface 1500 can be presented to the user subsequent to the user interface 500 (or the user interface 600) after the user selects the next icon on the input panel 508.

The pulse generation table 1502 can display pulse parameters, current measurements, and pulse delivery status. The pulse parameters displayed in the pulse generation table 1502 can be the same parameters displayed in the pulse parameters table 1002 displayed by the user interface 500 (or the user interface 600). In various embodiments, the pulse parameters displayed in the pulse generation table 1502 are sorted by voltage in descending order. The order of pulse parameters shown in the pulse generation table 1502 can be the order that pulses are delivered during the IRE procedure.

The user can modify the pulse parameters displayed in the pulse generation table 1502 and can activate or deactivate probe pairs before and after pulse delivery. The pulse generation table 1502 can display predicted current measurements for each probe pair following completion of a tissue conductivity test. The displayed predicted current measurements can be replaced with initial current measurements following initiation of pulse delivery.

The pulse generation table 1502 can also dynamically display maximum current values and change in current values for each probe pair throughout pulse delivery such that any change in value is updated and displayed. The pulse generation table 1502 can also dynamically display a count of the number of pulses delivered for each probe pair along with a status bar visually and/or textually indicating how many pulses have been delivered and/or remain to be delivered.

As shown in FIG. 15, certain parameters displayed in the pulse generation table 1502 can be provided based on parameters set by the user (or default values) listed in the pulse parameters table 1002 (e.g., pulse length, number of pulses, etc.). Other parameters—for example, max current, pulses delivered, etc.) do not provide values or do not provide non-zero values as these parameters can be provided in real-time during pulse delivery. Accordingly, the pulse generation table 1502 can display parameters set to initiate or plan an IRE procedure and parameters providing real-time monitoring of the IRE procedure. The user interface 1500 can dynamically update values for the parameters reflecting real-time monitoring data of the RE procedure.

As with parameters provided in the pulse parameters table 1002, parameters shown in the pulse generation table 1502 can be shown in similar manners to reflect default values, minimum and maximum values, or values set between a minimum and maximum value range. Further, these parameters can be modified by the user prior to implementing a planned IRE procedure. The user can also activate or deactivate probe pairs prior to implementing the planned IRE procedure.

The probe pair status grid 1504 can display a representation of the probe placement grid 502 displayed by the user interface 500 (or the user interface 600). Two of the of the probe grid icons 516 can change color between a first color (e.g., dark blue) and a second color (e.g., green) to indicate which probe pair in the selected probe array 510 is actively delivering pulses.

The electrical results chart 1506 allows the user to toggle between voltage, current, and resistance charts during and after pulse delivery. The user can select to display the voltage chart by selecting a voltage selection icon 1512. The user can select to display the current chart by selecting a current selection icon 1514. The user can select to display the resistance chart by selecting a resistance selection icon 1516. The charts can provide real-time data related to the pulses during delivery to allow the user to make modifications to pulse parameters during delivery.

In various embodiments, the electrical results chart 1506 can display results in various segments, with each segment corresponding to pulse delivery operations for a particular probe pair. For example, a first segment 1518 of the electrical results chart 1506 can correspond to pulse delivery provided by a first probe pair 1520 listed in the pulse generation table 1502. The first segment 1518 can display the electrical results—either voltage, current, or resistance results—related to pulse delivery for the first probe pair 1520 during and after delivery (e.g., real-time results during delivery or after completion of delivery).

Likewise, a second segment 1522 of the electrical results chart 1506 can correspond to pulse delivery provide by a second probe pair 1524 listed in the pulse generation table 1502. The second segment 1522 can display the electrical results—either voltage, current, or resistance results—related to pulse delivery for the second probe pair 1524 during and after delivery. In this way, the electrical results chart 1506 can display overall pulse delivery results while indicating which portions of the overall results relate to a specific probe pair.

In various embodiments, the electrical results chart 1506 can include one or more vertical grid lines 1528. The vertical grid lines 1528 can represent a transition between successive probe pairs listed in the pulse generation table 1502. As shown in FIG. 15, the vertical grid line 1528 indicates a transition from the first segment 1518 to the second segment 1522. Accordingly, the vertical grid line 1528 indicates when electrical results from the first probe pair 1520 end and the electrical results for the second probe pair 1524 begin.

In various embodiments, the electrical results chart 1506 can include a status bar 1526. The status bar 1526 can visually (and/or textually) indicate a status of pulse delivery. For example, the status bar 1526 can indicate an amount of pulses out of a total number of planned pulses for all probe pairs that have been successfully delivered. In contrast to the status indicator provided in the pulse generation table 1502 which can provide a status of pulses delivered for a specific probe pair only, the status bar 1526 indicates the status of pulse delivery across all probe pairs (e.g., the overall progress through pulse delivery and/or percentage complete).

The voltage gauge 1508 can display an amount of voltage available (e.g., stored on one or more capacitors of the IRE system) before, during, and after pulse delivery. The pulse delivery control panel 1510 can display options to the user for stopping pulse delivery, skipping a probe pair during pulse delivery, and charging or discharging stored voltage and is described further herein.

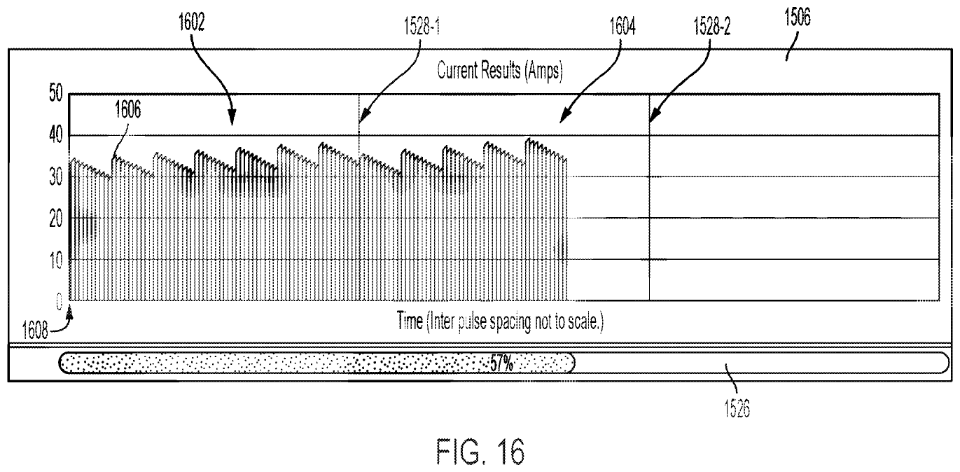

FIG. 16 illustrates an example display provided by the electrical results chart 1506 during pulse delivery. As shown in FIG. 16, two vertical grid lines 1528-1 and 1528-2 are shown to indicate that pulses from three different probe pairs will be displayed. A first set of pulses 1602 corresponding to the first probe pair are displayed and a second set of pulses 1604 corresponding to the second probe pair are also displayed. The first and second set of pulses 1602 and 1604 can be displayed in real-time—for example, each individually provided pulse can be displayed at substantially the same time as when the pulse is delivered to allow real-time monitoring of pulse delivery.

As an example, FIG. 16 shows the electrical results chart 1506 displaying current values (e.g., in Amps) for each delivered pulse. For purposes of discussion, a single delivered current pulse 1606 is designated for reference. The electrical results chart 1506 can include a scale 1608 representing possible current values for the delivered pulses such as, for example, the pulse 1606. Accordingly, each individual bar can represent a delivered pulse (e.g., the pulse 1606) and a height of each individual bar can indicate a corresponding current value for the delivered pulse. Delivered pulses can be shown in sequence in the electrical results chart 1506 from left to right (relative to the orientation of the electrical results chart 1506 shown in FIG. 16). A group of delivered pulses can represent a pulse train or portion thereof. A user can interact with the electrical results chart 1506 to zoom in on a pulse train or an individual pulse. Similar results—for example, in terms of voltage values (e.g., in Volts) and resistance values (e.g., in Ohms)—can be displayed by the electrical results chart 1506 for each individual delivered pulse based on the user selecting one of the icons 1512 or 1516, respectively.

As shown in FIG. 16, all pulses within the first set of pulses 1602 have been provided while additional pulses within the second set of pulses 1604 remain to be delivered. The status bar 1526, which can provide an indication of the overall pulse delivery progress, can show that a portion of the pulses for the second set of probes and all of the pulses for a third set of probes have yet to be delivered. The status bar 1526 can visually indicate how many pulses overall have been successfully delivered. A percentage of successfully provided pulses can also be displayed textually. In various embodiments, a color of the electrical results chart 1506 can change (e.g., from a first color to a second color) to indicate that the electrical results chart 1506 is displaying real-time results as pulses are being delivered (as opposed to showing results after all pulses have been delivered). The presentation of the electrical results chart 1506 as shown in FIG. 16 can quickly convey to the user how much of a treatment remains (e.g., in terms of time duration and/or number of pulses) and if the treatment pulses have been successfully delivered.

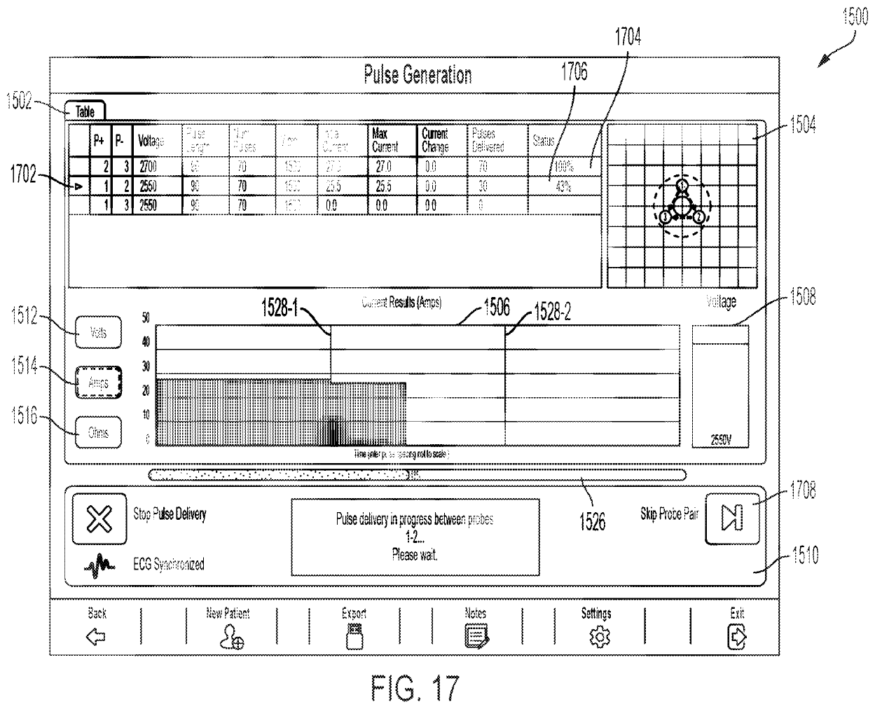

FIG. 17 illustrates the user interface 1500 during pulse delivery. As shown, an icon 1702 can indicate which probe pair listed in the pulse generation table 1502 is currently delivering pulses. Since the first probe pair has completed delivery, a first status indicator 1704 can provide an indication that delivery is complete. In various embodiments, the first status indicator 1704 can indicate how many planned pulses were successfully delivered (e.g., graphically and/or textually). In various embodiments, the first status indicator 1704 can be a status bar. Further, since the second probe pair is currently delivering pulses, a second status indicator 1706 can provide an indication of a percentage of pulses successfully delivered by the second probe pair. In this way, the user interface 1500 can quickly convey to the user which probe pair is currently delivering pulses (and an indication of how many pulses have been delivered and, indirectly, an indication of how many pulses remain to be delivered). As further shown in FIG. 17, the status bar 1526 can provide an indication of a percentage of all pulses successfully delivered across all probe pairs.

In various embodiments, a planned IRE procedure can be performed or implemented by generating and delivering pulses between all active probe pairs listed in the pulse generation table 1502 based on the pulse parameters set by the user (with certain parameters textually displayed in the pulse generation table 1502). The electrical results chart 1506 can be graphically updated during delivery of the pulses and after completion of the delivery of the pulses. During and after delivery, certain parameters listed in the pulse generation table 1502 can be updated based on monitoring by the IRE system which can provide delivery data to the user interface system 102.

As described herein, the user interface 1500 provides a dynamic display of information to reflect real-time pulse delivery by the IRE system. The pulse generation table 1502 can be dynamically updated with data values determined during pulse delivery. The voltage gauge 1508 can also be dynamically updated to reflect a charging status during pulse delivery. The electrical results chart 1506, as described herein, can be dynamically updated to reflect delivery of additional pulses by dynamically adding new pulses. The status bar in the pulse generation table 1502 and the status bar 1526 can be dynamically updated to indicate progress in delivering pulses for a specific active probe pair and a total of all planned pulses, respectively. As a result, the user interface 1500 advantageously provides the user with a dynamic visual representation of various key treatment parameters together at the same time, thereby obviating the need for the user to locate such information across various screens that may show only a portion of all the information provided on the user interface 1500.

The user interface 1500 can display a skip probe pair icon 1708 during pulse delivery. The user can select the skip probe pair icon 1708 to skip any remaining pulses to be delivered for the current active probe pair. The IRE system can then terminate pulse delivery through the current active probe pair. The IRE system, as indicated by the user interface 1500, can then advance to the next probe pair listed in the pulse generation table 1502 for pulse delivery.

In various embodiments, the user interface 1700 can provide user interfaces and/or displays to allow a user to stop pulse delivery entirely, pause a pulse delivery, resume a paused pulse delivery, repeat a pulse delivery (for all or one or more selected active probe pairs), or reset a pulse delivery (e.g., start a pulse delivery over).

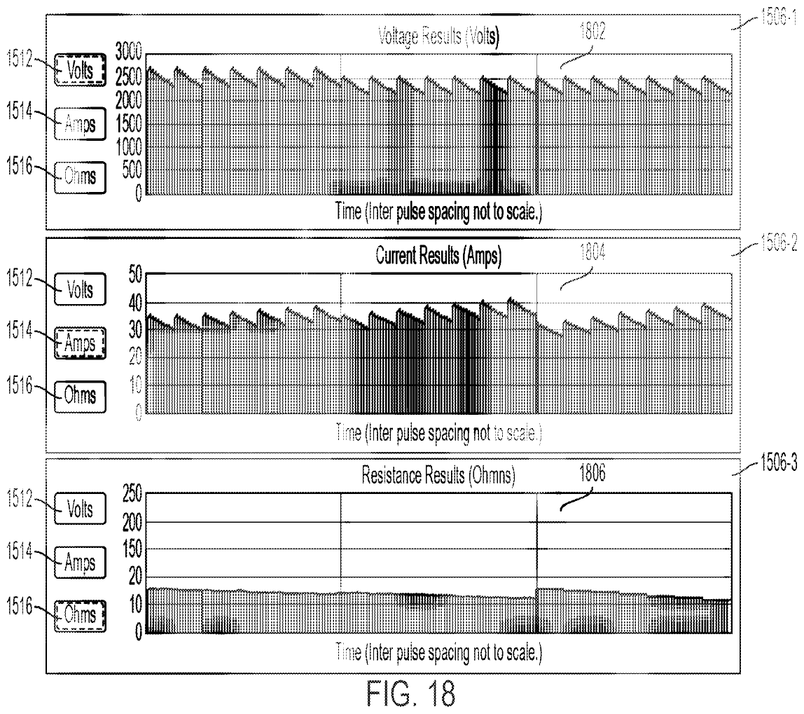

FIG. 18 illustrates example displays provided by the electrical results chart 1506 after pulse delivery. A first electrical results chart 1506-1 can display pulse voltage results 1802 when the user selects the volts icon 1512. A second electrical results chart 1506-2 can display pulse current results 1804 when the user selects the amps icon 1512. A third electrical results chart 1506-3 can display pulse resistance results 1806 when the user selects the ohms icon 1512. For each electrical results chart 1506-1 through 1506-3, full results for all pulses successfully delivered by all probes can be shown. Similar to the electrical results chart 1506 described in relation to FIG. 16, each of the electrical results charts 1506-1 through 1506-3 can display a vertical bar to represent a delivered pulse with the height of each pulse indicating a corresponding value (e.g., a voltage value, a current value, or a resistance value) for the pulse based on a provided scale. Further, the user can zoom into any portion of any of the electrical results charts 1506-1 through 1506-3 to view pulse delivery results in closer detail.

In various embodiments, the electrical results charts 1506-1 through 1506-3 can be stored in the memory 110 and/or shared with a remote device via the communications interface 102. As an example, the electrical results charts 1506-1 through 1506-3 can be used to consult with a physician, health care worker, or expert to determine if the completed treatment was successful or if a new treatment is required, along with how pulse and/or treatment parameters may be modified to result in a desired outcome.

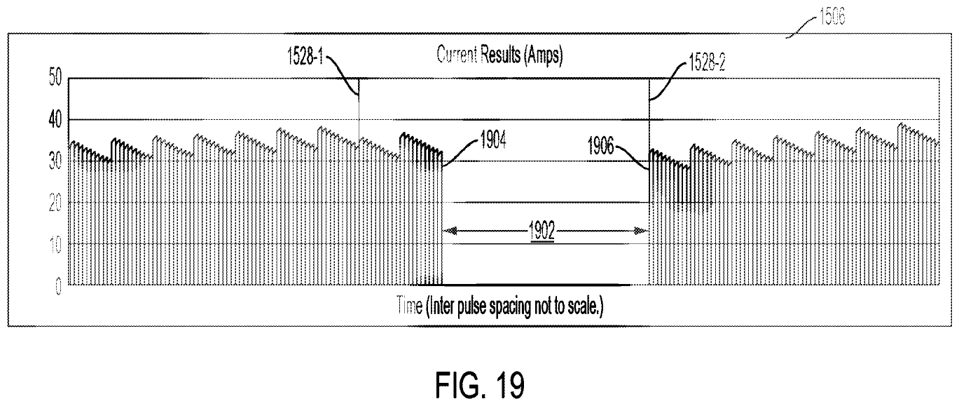

FIG. 19 illustrates an example display provided by the electrical results chart 1506 when a probe pair is skipped during pulse delivery. At any time during pulse delivery, the user can select the skip probe icon 1708 to indicate that the remaining pulses for the current active probe pair are to be skipped (e.g., not delivered). Pulse delivery for the current active probe pair can then stop. Pulse delivery can then begin for a next probe pair listed in the pulse generation table 1502. The electrical results chart 1506 can display a gap 1902 representing the skipped pulses. The gap 1902 can be shown between a last delivered pulse 1904 for the skipped probe pair and a first delivered pulse 1906 for the next probe pair.

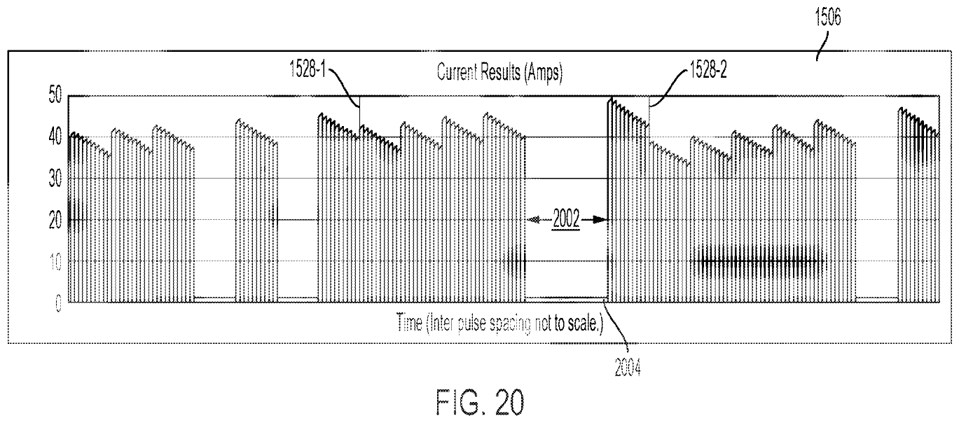

FIG. 20 illustrates an example display provided by the electrical results chart 1506 when a high current condition is detected. When a high current condition (e.g., overcurrent condition) is detected during pulse delivery, certain planned pulses may not be delivered. After a short delay (e.g., 5 seconds), when the high current condition expires, pulse delivery can resume. As shown in FIG. 20, the electrical results chart 1506 can display a gap 2002 with a corresponding indicator line 2004 on the horizontal axis of the electrical results chart 1506 to represent the detected overcurrent condition (e.g., when pulses were not delivered). The indicator line 2004 can be displayed in a highly visible color (e.g., orange) to indicate that the gap 2002 is due to an overcurrent condition. Overcurrent conditions can be represented with the gap 2002 and the indicator line 2004 during pulse delivery in the electrical results chart 1506 and after completion of pulse delivery in the electrical results chart 1506.

In various embodiments, one or more audible indicators or signals can be provided by the user interface system 100 when an overcurrent condition is detected. In various embodiments, a low current condition can also be detected and indicated by the electrical results chart 1506. Similar to the example display shown in FIG. 20, a low current condition can be similarly indicated (e.g., with a gap and an indicator line) but with a differently colored indicator line 2004.

A high current and/or an overcurrent condition can occur for a variety of reasons including, for example, when the probes are converging or the electrode tips of the probes are touching; when an electrode exposure setting is too large for the targeted tissue; when inter-probe spacing distances are incorrectly entered into the probe placement grid 502; when the voltage is set too high for the targeted tissue; and/or when the pulse length is set too large for the targeted tissue. In various embodiments, when a high current and/or an overcurrent condition occurs, the user interface system 100 can provide the user with a reason for the detected overcurrent condition.

In various embodiments, when an overcurrent condition or a low current condition is detected, the user can be provided various options (e.g., through a pop-up window) including, for example, the ability to stop all delivery, the ability to skip delivery for the currently active probe pair, the ability to redo pulse delivery for the currently active probe, the ability to redo delivery for all active probe pairs, and the ability to pause delivery (and possibly resume delivery).

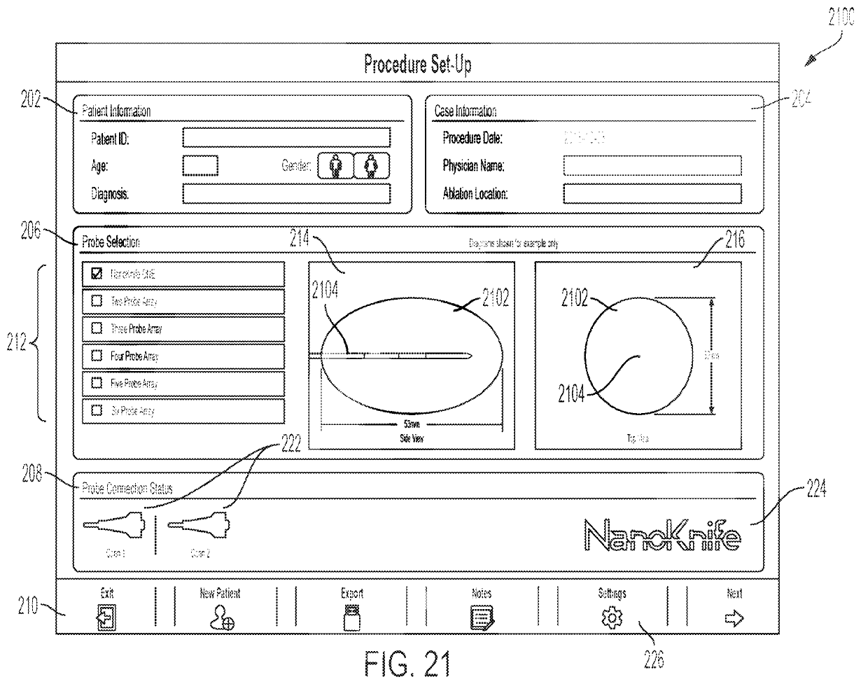

FIG. 21 illustrates a user interface 2100. The user interface 2100 can be provided by the user interface system 100. The user interface 2100 can represent a variation of the user interface 200 when the user selects a probe array that is different from the probe army selected by the user in relation to the user interface 200.

As shown in FIG. 21, the probe type list 212 can include and can indicate that a single probe (e.g., a single insertion device) has been selected. Accordingly, the first image pane 214 can show a side view of an ablation zone 2102 and a probe 2104 corresponding to the selected single probe from the probe type list 212. The second image pane 216 can show a top view of the ablation zone 2102 and the probe 2104 in accordance with the selected single insertion device. A size and shape of the ablation zone 2102 can vary from the size and shape of the ablation zone 218. The arrangement of the probe 2104 can vary from the number and arrangement of the probes 220. FIG. 21 provides another example of the user interface system 100 dynamically updating the first and second image panes 214 and 216 based on the user's selection of the probe array from the probe type list 212.

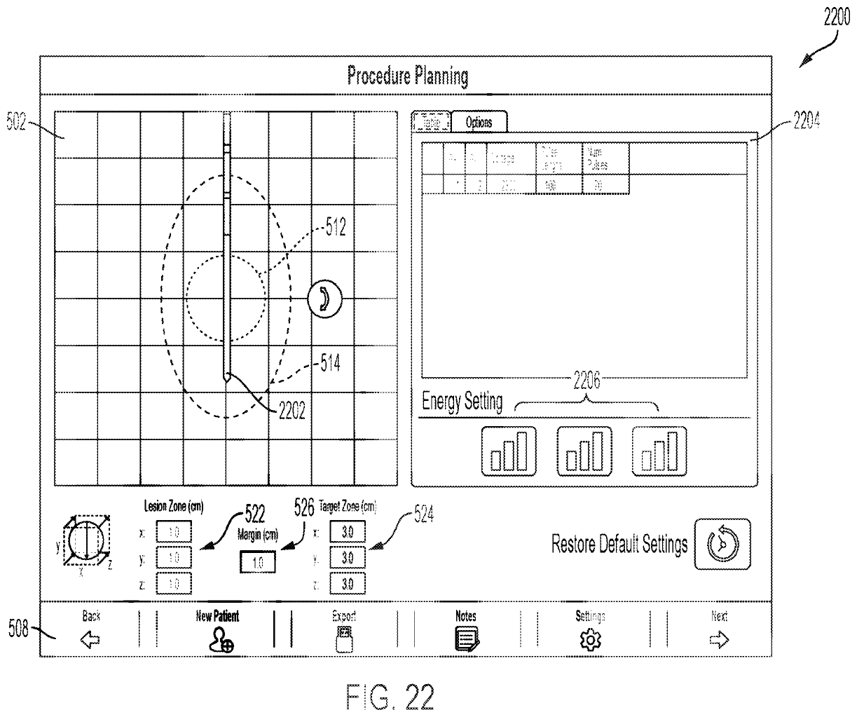

FIG. 22 illustrates a user interface 2200. The user interface 2200 can be provided by the user interface system 100. The user interface 2200 can represent a variation of the user interface 500 and can indicate a probe selection that is different from the probe selection displayed by the user interface 500.

As shown in FIG. 22, the probe placement grid 502 displays a single probe 2202, corresponding to the single probe selected from the probe type list 212 on the user interface 2100. The single probe 2202 can be an SID that includes two electrodes. The user interface 2200 can include all of the features and functionalities with regards to translation or rotation of the lesion zone 512 and the target ablation zone 514 described in relation to the user interface 500.

In contrast to the user interface 500, the user interface 2200 can display a table 2204 with pulse delivery parameters that are predetermined. The predetermined parameters can be selected from a set of available predetermined parameters, selectable by the user using a set of corresponding icons 2206. In various embodiments, pulse delivery parameters for the user interface 2200 can correspond to the available pulse parameters settings provided by the user interface 500 for probe arrays consisting of two or more probes. As an example, the icons 2206 can each specify a different predetermined number of pulses, pulse lengths, and voltages to apply for a treatment and, when selected, can be correspondingly displayed in the table 2204. The icons 2206 can also each specify different numbers of pulse sequences. For example, one of the icons 2206 can correspond to a low energy setting that specifies only a single set of pulses to be delivered between the electrodes of the SID 2202. Another one of the icons 2206 can correspond to a medium energy setting that specifies two sets of pulses to be delivered between the electrodes of the SID 2202. A third one of the icons 2206 can correspond to a high energy setting that specifies four sets of pulses to be delivered between the electrodes of the SID 2202. The icons 2206 can be considered to be predetermined pulse delivery and/or energy setting icons corresponding to predetermined treatment plans.

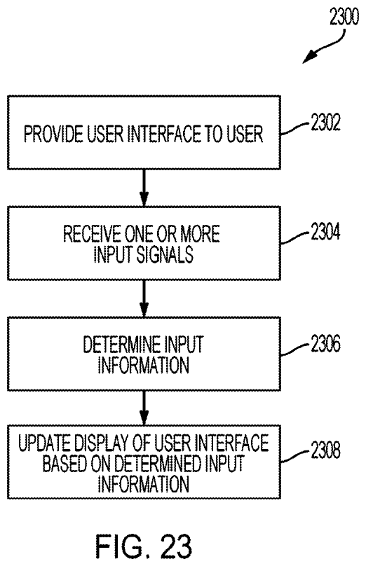

FIG. 23 illustrates an embodiment of a logic flow 2300 for providing any of the user interfaces described herein. The logic flow 2300 may be representative of some or all of the operations executed by one or more embodiments described herein. As an example, the logic flow 2300 can be implemented by the user interface system 100 to provide any of the user interfaces and/or displays depicted in FIGS. 2-22.

At 2302, a user interface can be provided to a user. The user interface can be any of the user interfaces or related displays described herein. The user interface may be displayed on a display device. The display device may be a touchscreen. The user interface can include a graphical portion for displaying information and/or for receiving input information. A control module, operable on a processor, may cause the display device to display the user interface. The control module may specify graphics or other visual elements stored in a memory for display on the display device.

At 2304, one or more input signals can be received. In various embodiments, the input signals can originate locally—for example, from local user input provided through a touchscreen or other user input device. In various embodiments, the input signals can originate remotely—for example, from a remote device in communication with a local device implementing the logic flow 1500 (e.g., an IRE system). An input signal receiver, operable on a processor, may be configured to receive the one or more input signals.

At 2306, input information from the input signals can be determined. The control module may be configured to determine the input information from the one or more input signals. The input information may include instructions and/or data values provided by the user. In various embodiments, the input information may include information provided by the IRE system—for example, information related to a status of one or more connected probes or information regarding delivery of pulses.

At 2308, the user interface can be adjusted based on the received input information. The control module can direct the display device to adjust the provided display to provide a dynamically updated user interface responsive to received input information. As an example, the user interface 500 can be modified based on user input to reflect any changes to a polarity assignment of a probe pair within the probe placement grid 502 and/or the pulse parameters table 1002.

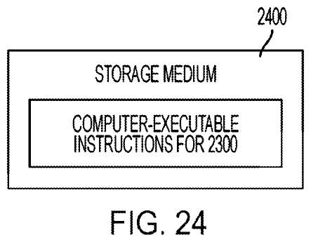

FIG. 24 illustrates an embodiment of a storage medium 2400. Storage medium 2400 may comprise any non-transitory computer-readable storage media or machine-readable storage media, such as an optical, magnetic or semiconductor storage media. In various embodiments, the storage medium 2400 may comprise an article of manufacture. In some embodiments, storage medium 2400 may store computer-executable instructions, such as computer-executable instructions to implement logic flow 2300 of FIG. 23.

Examples of a computer-readable storage medium or machine-readable storage medium may include any tangible media capable of storing electronic data, including volatile memory or non-volatile memory, removable or non-removable memory, erasable or non-erasable memory, writeable or re-writeable memory, and so forth. Examples of computer-executable instructions may include any suitable type of code, such as source code, compiled code, interpreted code, executable code, static code, dynamic code, object-oriented code, visual code, and the like. The storage medium 2400 may include instructions to be executed by the processor 108 for implementing the user interfaces described herein. The embodiments are not limited in this context.

The user interfaces and/or displays described herein and depicted in FIGS. 2-22 can include any textual and/or graphical depictions of an interface for techniques for controlling and/or monitoring an IRE system. The user interfaces can use any coloring, shading, and diminished or muted contrast as part of any textual and/or graphical depictions. The textual and/or graphical depictions can be dynamically updated and/or modified to reflect input from the user or information provided by the IRE system.

The following first set of examples pertain to further embodiments.

Example 1 is an irreversible electroporation (IRE) system comprising a processor operable with a memory and a display device, one or more input devices, an input signal receiver operable on the processor to receive one or more input signals from the one or more input devices, and a display controller operable on the processor to receive input information from the input signal receiver and to retrieve user interface information from the memory based upon the input information for display of a user interface on the display device, the user interface including a pulse parameters table for textual display of numerical values indicating a voltage, a pulse length, a number of pulses, and a distance for an active probe pair of a user selected probe array, the pulse parameter table further indicating a polarity of the active probe pair, and a probe placement grid for graphical display of the user selected probe array in relation to a lesion zone and a target ablation zone, each probe of the user selected probe array comprising a probe grid icon and a probe number, the active probe pair of the user selected probe array indicated by a dashed line connecting the probe grid icons of the active probe pair, the polarity of the active probe pair indicated by an arrow of the dashed line pointing to a negative probe of the active probe pair, the distance between the active probe pair indicated by a distance value positioned over the dashed line and by a spacing between the probe grid icons of the active probe pair.

Example 2 is an extension of Example 1 or any other example disclosed herein, the display controller operable to adjust graphical display of the arrow of the dashed line based on changes by a user to the polarity of the active probe pair in the pulse parameters table.

Example 3 is an extension of Example 1 or any other example disclosed herein, the display controller operable to adjust graphical display of the spacing between the probe grid icons of the active probe pair based on changes by a user to the numerical value of the distance of the active probe pair in the pulse parameters table.

Example 4 is an extension of Example 3 or any other example disclosed herein, the display controller operable to adjust the distance value positioned over the dashed line for the active probe pair based on changes by the user to the numerical value of the distance for the active probe pair in the pulse parameters table.

Example 5 is an extension of Example 1 or any other example disclosed herein, the user interface to further include a target ablation area settings panel for textual display of numerical values for a size of the lesion zone and a size of the target ablation zone.

Example 6 is an extension of Example 5 or any other example disclosed herein, the display controller operable to adjust graphical display of the lesion zone within the probe placement grid based on changes by a user to the numerical value for the size of the lesion zone.

Example 7 is an extension of Example 6 or any other example disclosed herein, the target ablation area setting panel further including textual display of a numerical value for a margin indicating a distance between an outer perimeter of the lesion zone and an outer perimeter of the target ablation zone, the display controller operable to adjust graphical display of the lesion zone and the target ablation zone based on changes by the user to the numerical value for the margin.

Example 8 is an extension of Example 7 or any other example disclosed herein, the display controller operable to adjust graphical display of a positioning of the user selected probe array relative to a positioning of the lesion zone and the target ablation zone based on an input from the user.

Example 9 is an extension of Example 7 or any other example disclosed herein, the display controller operable to adjust graphical display of a rotation of the lesion zone and the target ablation zone based on an input from the user.

Example 10 is an extension of Example 1 or any other example disclosed herein, the display controller operable to adjust graphical display of a color of the probe grid icon based on an operational status of a probe corresponding to the probe grid icon.

Example 11 is a computer-implemented method for controlling a user interface of an irreversible electroporation (IRE) system comprising receiving, by an input signal receiver operable on a processor, one or more input signals from one or more input devices, receiving, by a display controller operable on the processor, input information based upon the one or more input signals, and displaying, by the display controller operable on the processor, a user interface on a display device, the user interface including a pulse parameters table for textual display of numerical values indicating a voltage, a pulse length, a number of pulses, and a distance for an active probe pair of a user selected probe array, the pulse parameter table indicating a polarity of the active probe pair and a probe placement grid for graphical display of the user selected probe array in relation to a lesion zone and a target ablation zone, each probe of the user selected probe array comprising a probe grid icon and a probe number, the active probe pair of the user selected probe array indicated by a dashed line connecting the probe grid icons of the active probe pair, the polarity of the active probe pair indicated by an arrow of the dashed line pointing to a negative probe of the active probe pair, the distance between the active probe pair indicated by a distance value positioned over the dashed line and by a spacing between the probe grid icons of the active probe pair.

Example 12 is an extension of Example 11 or any other example disclosed herein, further comprising adjusting, by the display controller operable on the processor, graphical display of the arrow of the dashed line based on changes by a user to the polarity of the active probe pair in the pulse parameters table.

Example 13 is an extension of Example 11 or any other example disclosed herein, further comprising adjusting, by the display controller operable on the processor, graphical display of the spacing between the probe grid icons of the active probe pair based on changes by a user to the numerical value of the distance of the active probe pair in the pulse parameters table.

Example 14 is an extension of Example 13 or any other example disclosed herein, further comprising adjusting, by the display controller operable on the processor, the distance value positioned over the dashed line for the active probe pair based on changes by the user to the numerical value of the distance for the active probe pair in the pulse parameters table.

Example 15 is an extension of Example 11 or any other example disclosed herein, further comprising adjusting, by the display controller operable on the processor, graphical display of the user selected probe array based on a user adding an additional active probe pair to the pulse parameters table.

Example 16 is an article comprising a non-transitory computer-readable storage medium including instructions that, when executed by a processor, enable an irreversible electroporation (IRE) system to receive, by an input signal receiver operable on a processor, one or more input signals from one or more input devices, receive, by a display controller operable on the processor, input information based upon the one or more input signals, and display, by the display controller operable on the processor, a user interface on a display device, the user interface including a pulse parameters table for textual display of numerical values indicating a voltage, a pulse length, a number of pulses, and a distance for an active probe pair of a user selected probe array, the pulse parameter table indicating a polarity of the active probe pair and a probe placement grid for graphical display of the user selected probe array in relation to a lesion zone and a target ablation zone, each probe of the user selected probe array comprising a probe grid icon and a probe number, the active probe pair of the user selected probe array indicated by a dashed line connecting the probe grid icons of the active probe pair, the polarity of the active probe pair indicated by an arrow of the dashed line pointing to a negative probe of the active probe pair, the distance between the active probe pair indicated by a distance value positioned over the dashed line and by a spacing between the probe grid icons of the active probe pair.

Example 17 is an extension of Example 16 or any other example disclosed herein, wherein the display controller operable on the processor adjusts graphical display of the arrow of the dashed line based on changes by a user to the polarity of the active probe pair in the pulse parameters table.

Example 18 is an extension of Example 16 or any other example disclosed herein, wherein the display controller operable on the processor adjusts graphical display of the spacing between the probe grid icons of the active probe pair based on changes by a user to the numerical value of the distance of the active probe pair in the pulse parameters table.

Example 19 is an extension of Example 18 or any other example disclosed herein, wherein the display controller operable on the processor adjusts graphical display of the distance value positioned over the dashed line for the active probe pair based on changes by the user to the numerical value of the distance for the active probe pair in the pulse parameters table.

Example 20 is an extension of Example 16 or any other example disclosed herein, wherein the display controller operable on the processor adjusts graphical display of the user selected probe array based on a user adding an additional active probe pair to the pulse parameters table.

Example 21 is an irreversible electroporation (IRE) system, comprising a processor operable with a memory and a display device, one or more input devices, an input signal receiver operable on the processor to receive one or more input signals from the one or more input devices, and a display controller operable on the processor to receive input information from the input signal receiver and to retrieve user interface information from the memory based upon the input information for display of a user interface on the display device, the user interface including a pulse parameters table for textual display of numerical values indicating a voltage, a pulse length, a number of pulses, and a distance for an active probe pair of a user selected probe array, the pulse parameter table further indicating a polarity of the active probe pair, and a probe placement grid for graphical display of the user selected probe array as specified textually in the pulse parameters table and in relation to a lesion zone and a target ablation zone.

The following second set of examples pertain to further embodiments.

Example 1 is an irreversible electroporation (IRE) system comprising a processor operable with a memory and a display device, one or more input devices, an input signal receiver operable on the processor to receive one or more input signals from the one or more input devices, and a display controller operable on the processor to receive input information from the input signal receiver and to retrieve user interface information from the memory based upon the input information for display of a user interface on the display device, the user interface including a pulse generation table for textual display of numerical values indicating a voltage, a pulse duration, and a number of pulses for pulse delivery by an active probe pair of a user selected probe array, a probe pair status grid for graphical display of the user selected probe array in relation to a lesion zone and a target ablation zone, and an electrical results chart for graphical display of the pulse delivery by the active probe pair based on the numerical values for the voltage, the pulse duration, and the number of pulses for the active probe pair within the pulse generation table.

Example 2 is an extension of Example 1 or any other example disclosed herein, the display controller operable to adjust graphical display of the electrical results chart in real-time as the pulse delivery by the active probe pair is performed.

Example 3 is an extension of Example 1 or any other example disclosed herein, the display controller operable to adjust graphical display of the electrical results chart after the pulse delivery by the active probe pair is performed.

Example 4 is an extension of Example 1 or any other example disclosed herein, the display controller operable to adjust graphical display of the electrical results chart to include voltage results based on an input of a user.

Example 5 is an extension of Example 1 or any other example disclosed herein, the display controller operable to adjust graphical display of the electrical results chart to include current results based on an input of a user.

Example 6 is an extension of Example 1 or any other example disclosed herein, the display controller operable to adjust graphical display of the electrical results chart to include resistance results based on an input of a user.

Example 7 is an extension of Example 1 or any other example disclosed herein, the display controller operable to adjust graphical display of the user selected probe array within the probe pair status grid to indicate the active probe pair is performing pulse delivery.

Example 8 is an extension of Example 1 or any other example disclosed herein, the electrical results chart to include a vertical grid line to indicate a transition of pulse delivery between successive probe pairs listed in the pulse generation table.

Example 9 is an extension of Example 1 or any other example disclosed herein, the display controller operable to display an icon in the pulse generation table to indicate the active probe pair performing pulse delivery.

Example 10 is an extension of Example 1 or any other example disclosed herein, the display controller operable to display, within the electrical results chart, a gap to indicate skipped pulses during the pulse delivery by the active probe pair.

Example 11 is an extension of Example 10 or any other example disclosed herein, the display controller operable to display, within the electrical results chart, an indicator line on a horizontal axis of the electrical results chart corresponding to the gap to indicate a detected overcurrent condition.

Example 12 is a computer-implemented method for controlling a user interface of an irreversible electroporation (IRE) system comprising receiving, by an input signal receiver operable on a processor, one or more input signals from one or more input devices, receiving, by a display controller operable on the processor, input information based upon the one or more input signals, and displaying, by the display controller operable on the processor, a user interface on a display device, the user interface including a pulse generation table for textual display of numerical values indicating a voltage, a pulse duration, and a number of pulses for pulse delivery by an active probe pair of a user selected probe array, a probe pair status grid for graphical display of the user selected probe array in relation to a lesion zone and a target ablation zone, and an electrical results chart for graphical display of the pulse delivery by the active probe pair based on the numerical values for the voltage, the pulse duration, and the number of pulses for the active probe pair within the pulse generation table.

Example 13 is an extension of Example 120 or any other example disclosed herein, further comprising adjusting, by the display controller operable on the processor, graphical display of the electrical results chart in real-time as the pulse delivery by the active probe pair is performed.

Example 14 is an extension of Example 12 or any other example disclosed herein, further comprising adjusting, by the display controller operable on the processor, graphical display of the electrical results chart after the pulse delivery by the active probe pair is performed.

Example 15 is an extension of Example 12 or any other example disclosed herein, further comprising adjusting, by the display controller operable on the processor, graphical display of the user selected probe array within the probe pair status grid to indicate the active probe pair is performing pulse delivery.

Example 16 is an extension of Example 15 or any other example disclosed herein, further comprising changing a color of probe grid icons corresponding to the active probe pair to indicate the active probe pair is performing pulse delivery.

Example 17 is an article comprising a non-transitory computer-readable storage medium including instructions that, when executed by a processor, enable an irreversible electroporation (IRE) system to receive, by an input signal receiver operable on a processor, one or more input signals from one or more input devices, receive, by a display controller operable on the processor, input information based upon the one or more input signals, and display, by the display controller operable on the processor, a user interface on a display device, the user interface including a pulse generation table for textual display of numerical values indicating a voltage, a pulse duration, and a number of pulses for pulse delivery by an active probe pair of a user selected probe array, a probe pair status grid for graphical display of the user selected probe array in relation to a lesion zone and a target ablation zone, and an electrical results chart for graphical display of the pulse delivery by the active probe pair based on the numerical values for the voltage, the pulse duration, and the number of pulses for the active probe pair within the pulse generation table.

Example 18 is an extension of Example 17 or any other example disclosed herein, wherein the display controller operable on the processor adjusts graphical display of the electrical results chart in real-time as the pulse delivery by the active probe pair is performed.

Example 19 is an extension of Example 17 or any other example disclosed herein, wherein the display controller operable on the processor adjusts graphical display of the electrical results chart after the pulse delivery by the active probe pair is performed.

Example 20 is an extension of Example 17 or any other example disclosed herein, wherein the display controller operable on the processor adjusts graphical display of the user selected probe array within the probe pair status grid to indicate the active probe pair is performing pulse delivery.

Example 21 is an irreversible electroporation (IRE) system comprising a processor operable with a memory and a display device, one or more input devices, an input signal receiver operable on the processor to receive one or more input signals from the one or more input devices, and a display controller operable on the processor to receive input information from the input signal receiver and to retrieve user interface information from the memory based upon the input information for display of a user interface on the display device, the user interface including a pulse parameters table for textual display of numerical values indicating a voltage, a pulse length, and a number of pulses for a single insertion device (SID), the SID comprising a single probe having two electrodes, and a probe placement grid for graphical display of the SID in relation to a lesion zone and a target ablation zone.

Example 22 is an extension of Example 21 or any other example disclosed herein, wherein the numerical values indicating the voltage, the pulse length, and the number of pulses is predetermined and non-adjustable.

Example 23 is an extension of Example 21 or any other example disclosed herein, the user interface further including a first energy icon selectable by the user corresponding to a single set of pulses to be delivered by the electrodes of the SID, a second energy icon selectable by the user corresponding to a two sets of pulses to be delivered by the electrodes of the SID, and a third energy icon selectable by the user corresponding to a four sets of pulses to be delivered by the electrodes of the SID.

Various embodiments described herein may comprise one or more elements. An element may comprise any structure arranged to perform certain operations. Each element may be implemented as hardware, software, or any combination thereof. Any reference to “one embodiment” or “an embodiment” means that a particular feature, structure, or characteristic described in connection with the embodiment is included in at least one embodiment. The appearances of the phrases “in one embodiment,” “in some embodiments,” and “in various embodiments” in various places in the specification are not necessarily all referring to the same embodiment.

In various instances, for simplicity, well-known operations, components, and circuits have not been described in detail so as not to obscure the embodiments. It can be appreciated that the specific structural and functional details disclosed herein may be representative and do not necessarily limit the scope of the embodiments. Certain embodiments of the present invention were described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

1.-20. (canceled)

21. A system comprising:
a generator coupled to a processor, the generator configured to deliver at least one electrical pulse to a target tissue;
a display device in communication with the processor; and
a memory in communication with the processor, the memory storing instructions that, when executed, cause the processor to:
receive a probe parameter input,
calculate an additional probe parameter based on the probe parameter input,
generate a first graphical representation of a plurality of probe icons based on at least the probe parameter input and the calculated additional probe parameter, and
display the first graphical representation on the display device.

22. The system of claim 21, wherein the probe parameter input comprises a distance measurement.

23. The system of claim 22, further comprising:
a plurality of probes operatively coupled to the generator, wherein each of the plurality of probe icons is a virtual representation of one of the plurality of probes, and wherein the distance measurement includes a first distance between a first pair of probes within the plurality of probes.

24. The system of claim 23, wherein the additional probe parameter includes a second distance between a second pair of probes within the plurality of probes.

25. The system of claim 21, wherein instructions, when executed, further cause the processor to:
generate a second graphical representation of an indication of an association between a pair of probe icons within the plurality of probe icons.

26. The system of claim 25, wherein the indication includes a graphic linking the pair of probe icons.

27. The system of claim 26, wherein the graphic includes a dashed line.

28. The system of claim 21, wherein the instructions, when executed, further cause the processor to:
receive a lesion information input, and
generate a second graphical representation of a target area based on at least the lesion information input.

29. The system of claim 28, wherein the instructions, when executed, further cause the processor to:
display the second graphical representation of the target area and the first graphical representation of the plurality of probe icons on the display device, wherein the first graphical representation is superimposed over the second graphical representation.

30. The system of claim 21, wherein the instructions, when executed, further cause the processor to:
monitor, in real-time, a pulse parameter relating to the at least one electrical pulse delivered to the target tissue by the generator.

31. The system of claim 30, wherein the instructions, when executed, further cause the processor to:
display the pulse parameter on the display device.

32. The system of claim 31, wherein the instructions, when executed, further cause the processor to:
modify the pulse parameter displayed on the display device in response to the real-time monitoring of the pulse parameter.

33. A system comprising:
a generator coupled to a processor, the generator configured to deliver at least one electrical pulse to a target tissue;
a display device in communication with the processor; and
a memory in communication with the processor, the memory storing instructions that, when executed, cause the processor to:
receive a lesion information input,
generate a first graphical representation of a target area based on at least the lesion information input,
receive a probe parameter input,
calculate an additional probe parameter based on the probe parameter input,
generate a second graphical representation of a plurality of probe icons based on at least the probe parameter input and the calculated additional probe parameter,
display the first graphical representation of the target area and the second graphical representation of the plurality of probe icons on the display device, wherein the second graphical representation is superimposed over the first graphical representation, and
monitor, in real-time, a pulse parameter relating to the at least one electrical pulse delivered to the target tissue by the generator.

34. The system of claim 33, wherein the probe parameter input is a distance measurement.

35. The system of claim 34, further comprising:
a plurality of probes operatively coupled to the generator, wherein each of the plurality of probe icons is a virtual representation of one of the plurality of probes, and wherein the distance measurement includes a first distance between a first pair of probes within the plurality of probes.

36. The system of claim 35, wherein the additional probe parameter includes a second distance between a second pair of probes within the plurality of probes.

37. The system of claim 33, wherein the instructions, when executed, further cause the processor to:
display the pulse parameter on the display device.

38. The system of claim 37, wherein the instructions, when executed, further cause the processor to:
modify the pulse parameter on the display device in response to the real-time monitoring of the pulse parameter.

39. A system comprising:
a generator coupled to a processor, the generator configured to deliver at least one electrical pulse to a target tissue;
a display device in communication with the processor; and
a memory in communication with the processor, the memory storing instructions that, when executed, cause the processor to:
receive a lesion information input,
generate a first graphical representation of a target area based on at least the lesion information input,
receive a probe parameter input,
generate a second graphical representation of a plurality of probe icons based on at least the probe parameter input,
display the first graphical representation of the target area and the second graphical representation of the plurality of probe icons on the display device, wherein the second graphical representation is superimposed over the first graphical representation, and
monitor, in real-time, a pulse parameter relating to the at least one electrical pulse delivered to the target tissue by the generator.

40. The system of claim 39, wherein the instructions, when executed, further cause the processor to:
calculate an additional probe parameter based upon the probe parameter input,
wherein the second graphical representation of the plurality of probe icons is further based on the additional probe parameter.
